(19) United States
(12) Patent Application Publication
(10) Pub. No.: US 2024/0093256 A1
(43) Pub. Date: Mar. 21, 2024

(54) Stabilized N-Terminally Truncated Terminal Deoxynucleotidyl Transferase Variants and Uses Thereof

(71) Applicant: DNA Script, Le Kremlin-Bicêtre (FR)

(72) Inventors: Mikhael Soskine, Franconville (FR); Elise Champion, Paris (FR); Daniel McHale, Le Kremlin-Bicêtre (FR)

(21) Appl. No.: 18/027,594

(22) PCT Filed: Sep. 22, 2021
(86) PCT No.: PCT/EP2021/076073

§ 371 Date: Aug. 21, 2023

(30) Foreign Application Priority Data
Sep. 22, 2020 (EP) 20306079.3
Mar. 19, 2021 (EP) 21305341.6

(51) Int. Cl. C12P 19/34; C12N 9/12

(52) U.S. Cl. CPC C12P 19/34; C12N 9/1264; C12Y 207/07031

(57) ABSTRACT

The present invention is directed to terminal deoxynucleotidyl transferase (TdT) variants from a variety of species which have been engineered for increase compactness and enhanced efficiency in incorporating reversibly blocked nucleoside triphosphates into a polynucleotide, especially polynucleotides attached porous solid supports. The invention includes use of such TdTs for synthesizing polynucleotides of any predetermined sequence.

### BACKGROUND

Synthetic polynucleotides of predetermined sequences are fundamental components in many different technologies, including molecular diagnostics, genomic and diagnostic sequencing, nucleic acid amplification, therapeutic antibody development, synthetic biology, nucleic acid-based therapeutics, DNA origami, DNA-based data storage, and the like. Recently, interest has arisen in supplementing or replacing chemically-based synthesis methods by enzymatically-based methods using template-free polymerases, such as, terminal deoxynucleotidyl transferase (TdT), because of the proven efficiency of such enzymes and the benefit of mild non-toxic reaction conditions, e.g. Godron et al, International patent publication, WO2020/120442; Champion et al, U.S. patent Ser. No. 10/435,676; Ybert et al, International patent publication WO2015/159023; Hiatt et al, U.S. Pat. No. 5,763,594; Jensen et al, Biochemistry, 57: 1821-1832 (2018); and the like. Most approaches in enzyme-based synthesis require the use of reversibly blocked nucleoside triphosphates in order to obtain a desired sequence in the polynucleotide product. Unfortunately, however, natural TdTs incorporate such modified nucleoside triphosphates with greatly reduced efficiency as compared to unmodified nucleoside triphosphates and are not designed to catalyze coupling reactions in solid supports.

In view of the above, the field of template-free enzymatically-based polynucleotide synthesis would be advanced if new template-free polymerases, such as variant TdTs, were available that could incorporate reversibly blocked nucleoside triphosphates with greater efficiency and that were sufficiently compact and stable to catalyze coupling reactions on the interior of solid supports and resins.

### SUMMARY OF THE INVENTION

The present invention is directed to N-terminally truncated terminal deoxynucleotidyl transferase (TdT) variants having a stabilizing mutation at the N-terminus of the truncated sequence. The present invention is also directed to compact TdT variants having a minimized size and radius of gyration which permits the TdT variants to access the interior spaces of solid supports and resins more readily than wild type TdTs or prior TdT variants. In some embodiments, the stabilizing mutation of the N-terminally truncated TdT variant is at position Q152 (where the amino acid position number is with respect to full-length mouse TdT (SEQ ID NO: 1)), or at a functionally equivalent position in other TdT amino acid sequences. In some embodiments, TdT variants of the invention comprise an interior affinity tag to facilitate purification and to minimize its radius of gyration. In some embodiments, TdT variants of the invention display enhanced efficiency in incorporating reversibly blocked nucleoside triphosphates into a polynucleotide, particularly when the polynucleotide is attached to a solid support.

In some embodiments, an N-terminally truncated TdT variant of the invention comprises an amino acid sequence at least ninety percent identical to an amino acid sequence selected from SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 44, wherein a glutamine at position 4 of such amino acid sequence is substituted with a stabilizing amino acid; and wherein the TdT variant (i) is capable of synthesizing a nucleic acid fragment without a template and (ii) is capable of incorporating a 3′-O-protected-nucleotide onto a free 3′-hydroxyl of a polynucleotide. In some embodiments, a stabilizing amino acid is selected from the group consisting of E, S, D and N. In other embodiments, the stabilizing amino acid is E.

In some embodiments, a stabilizing amino acid is a substitution for the glutamine at position 4 (or a functionally equivalent position) which increases the melting temperature of a TdT variant by at least 1° C. relative to a melting temperature of a TdT of the same amino acid sequence except for said stabilizing amino acid substitution, wherein the melting temperature is determined by a fluorescence-based thermal shift assay, which can be performed by using a SYPRO Orange dye.

In some embodiments, an N-terminally truncated TdT of the invention comprises an amino acid sequence at least ninety percent identical to an amino acid sequence of SEQ ID NO: 42, 44, 46, 48, 50, 78 or 80 wherein the amino acid sequence of the TdT variant comprises a stabilizing amino acid at position 4 with respect to SEQ ID NO: 44, 78 and 80 a stabilizing amino acid at position 5 with respect to SEQ ID NO: 42, and a stabilizing amino acid at position 6 with respect to SEQ ID NO: 46, 48 and 50. In further embodiments, an N-terminally truncated TdT variant of the invention comprises an amino acid sequence at least ninety percent identical to the amino acid sequence of SEQ ID NO: 42, wherein the amino acid at position 4 of the amino acid sequence of such TdT variant is a stabilizing amino acid. In still further embodiments, an N-terminally truncated TdT variant of the invention comprises an amino acid sequence at least ninety percent identical to the amino acid sequence of SEQ ID NO: 78 or 80, wherein the amino acid at position 4 of each amino acid sequence of such TdT variant is a stabilizing amino acid.

In some embodiments of the invention, the TdT variant has a loop 2 region, wherein the TdT variant comprises a peptide affinity tag inserted into the loop 2 region, and wherein the TdT variant (i) is capable of synthesizing a nucleic acid fragment without a template and (ii) is capable of incorporating a 3′-O-protected-nucleotide onto a free 3′-hydroxyl of a polynucleotide. In some embodiments, TdT variants of the invention further have an affinity tag disposed in its interior by swapping a segment of one or more amino acids in the loop 2 region of the TdT with a segment of amino acids comprising an affitity tag. In some embodiments, the segment of loop 2 swapped for a segment comprising the affinity tag are the same size. That is, the number of amino acids removed from loop 2 is equal to the number of amino acids including the affinity tag added back to loop 2. In some embodiments, the TdT variant of the invention comprises an amino acid sequence at least ninety percent identical to an amino acid sequence selected from SEQ ID NOs: 42, 44, 46, 48, or 50, wherein each of the amino acid sequences of SEQ ID NOs: 42, 44, 46, 48, and 50 have said peptide affinity tag inserted into said loop 2 region. In some embodiments, the TdT variant of the invention comprises an amino acid sequence at least ninety percent identical to an amino acid sequence selected from SEQ ID NOs: 57 or 58.

In some embodiments, the invention is directed to methods of using TdT variants of the invention to synthesize polynucleotides of any predetermined sequence. Said method may comprise the steps of: a) providing an initiator having a 3′-terminal nucleotide having a free 3′-hydroxyl; b) b) repeating cycles of (i) contacting under elongation conditions the initiator or elongated fragments having free 3′-O-hydroxyls with a 3′-O-blocked nucleoside triphosphate and a TdT variant according to any one of claims 1 to 11, so that the initiator or elongated fragments are elongated by incorporation of a 3′-O-blocked nucleoside triphosphate to form 3′-O-blocked elongated fragments, and (ii) deblocking the elongated fragments to form elongated fragments having free 3′-hydroxyls, until the polynucleotide is formed. In some embodiments, an N-terminally truncated terminal deoxynucleotidyl transferase (TdT) variant of the invention is provided as isolated protein.

In some embodiments, the percent identity value with respect to each of the foregoing TdT variants is at least 80 percent identity with the indicated SEQ ID NOs; in some embodiments, the above percent identity value is at least 90 percent identity with the indicated SEQ ID NOs; in some embodiments, the above percent identity value is at least 95 percent identity with the indicated SEQ ID NOs; in some embodiments, the above percent identity value is at least 97 percent identity; in some embodiments, the above percent identity value is at least 98 percent identity; in some embodiments, the above percent identity value is at least 99 percent identity. As used herein, the percent identity values used to compare a reference sequence to a variant sequence do not include the expressly specified amino acid positions containing substitutions of the variant sequence; that is, the percent identity relationship is between sequences of a reference protein and sequences of a variant protein outside of the expressly specified positions containing substitutions in the variant. Thus, for example, if the reference sequence and the variant sequence each comprised 100 amino acids and the variant sequence had mutations at positions 25 and 81, then the percent homology would be in regard to sequences 1-24, 26-80 and 82-100. In regard to (ii) above, in some embodiments, such 3′-O-modified nucleotide may comprise a 3′-O—NH2-nucleoside triphosphate, a 3′-O-azidomethyl-nucleoside triphosphate, a 3′-O-allyl-nucleoside triphosphate, a 3′-O-(2-nitrobenzyl)-nucleoside triphosphate, a 3′-O-nitro-nucleoside triphosphate or a 3′-O-propargyl-nucleoside triphosphate. In other embodiments, such 3′-O-modified nucleotide may comprise a 3′-O—NH2-nucleoside triphosphate or a 3′-O-azidomethyl-nucleoside triphosphate.

The invention further relates to the use of a TdT variant of the invention for synthesizing a nucleic acid molecule without template by the successive addition of one or more 3′-O-modified nucleotides to a nucleic acid fragment. In some embodiments, such methods comprise the steps of (a) providing an initiator, usually attached to a solid support, having a free 3′-hydroxyl; (b) reacting under enzymatic extension conditions a TdT variant of the invention with the initiator or an extended initiator in the presence of a 3′-O-reversibly blocked nucleoside triphosphate. In some embodiments, such method further includes steps of (c) deblocking the extended initiators to form extended initiators with free 3′-hydroxyls and (d) repeating steps (b) and (c) until a nucleic acid molecule of a predetermined sequence is synthesized.

In further embodiments, the invention includes nucleic acid molecules encoding a variant TdTs described above, expression vectors comprising such nucleic acid molecules, and host cells comprising the aforementioned nucleic acid molecules or the aforementioned expression vectors. In still further embodiments, the invention includes processes for producing a variant TdT of the invention, wherein a host cell is cultivated under culture conditions allowing the expression of the nucleic acid encoding said variant TdT, and wherein the variant TdT is optionally retrieved. The invention also includes kits for performing template-free polynucleotide elongations of any predetermine sequence, wherein the kits include a TdT variant of the invention. Such kits may further comprise 3′-O-blocked deoxyribonucleoside triphosphates (dNTPs) for A, C, G and T for DNA elongation, or 3′-O-blocked ribonucleoside triphosphates (rNTPs) for rA, rC, rG and U for RNA elongation.

The present invention advantageously overcomes problems in the field of template-free enzymatic nucleic acid synthesis, particularly synthsis on porous solid supports, by providing new N-terminally truncated TdT variants which have smaller physical size, thereby minimizing steric interference of enzyme access to growing chains near a solid surface or in the interior of a solid support. In some embodiments, steric interference is further reduced by providing TdT variants having interior affinity tags rather than N-terminal or C-terminal affinity tags which serve to increase the enzymes radius of gyration thereby reducing its compactness and ability to access interior spaces of porous solid supports.

### BRIEF DESCRIPTION OF THE DRAWINGS

FIG. 1 illustrates diagrammatically the steps of a method of template-free enzymatic nucleic acid synthesis using TdT variants of the invention.

### DETAILED DESCRIPTION OF THE INVENTION

While the invention is amenable to various modifications and alternative forms, specifics thereof have been shown by way of example in the drawings and will be described in detail. It should be understood that the intention is not to limit the invention to the particular embodiments described. It is the intention to cover all modifications, equivalents, and alternatives falling within the spirit and scope of the invention. Guidance for aspects of the invention is found in many available references and treatises well known to those with ordinary skill in the art, including, for example, Sambrook et al. (1989), Molecular cloning: A Laboratory Manual, Cold Spring Harbor Laboratory, and the like.

The present invention provides N-terminally truncated and stabilized variants of the TdT polymerase that can be used for synthesizing polynucleotides, such as DNA or RNA, of predetermined sequences without the use of template strand. The TdT variants of the invention allow modified nucleotides, and more particularly 3′O-reversibly blocked nucleoside triphosphates, to be used in an enzyme-based method of polynucleotide synthesis.

In some embodiments, TdT variants of the invention are derived from natural TdTs such as those listed in Table 1 with a substitution at one or more, preferable two or more, of the indicated amino acid positions in addition to the stabilizing substitution of the glutamine at position 4. In other embodiments, TdT variants of the invention are derived from natural TdTs such as those listed in Table 1 with a substitution at every one of the indicated amino acid positions in addition to the stabilizing substitution of the glutamine at position 4.

TABLE 1

| SEQ ID NO | Animal | Substitutions | | | | |
|---|---|---|---|---|---|---|
| 1 | Mouse | M192R/Q | C302G/R | R336L/N | R454P/N/A/V | E457N/L/T/S/K |
| 2 | Mouse | M44R/Q | C154G/R | R188L/N | R306P/N/A/V | E309N/L/T/S/K |
| 3 | Bovine | M44R/Q | C154G/R | R188L/N | R305P/N/A/V | E308N/L/T/S/K |
| 4 | Human | M44R/Q | C154G/R | R188L/N | R305P/N/A/V | E308N/L/T/S/K |
| 5 | Chicken | — | C154G/R | R188L/N | R302P/N/A/V | — |
| 6 | Possum | M44R/Q | C154G/R | R188L/N | R312P/N/A/V | E315N/L/T/S/K |
| 7 | Shrew | M44R/Q | C154G/R | R188L/N | — | E309N/L/T/S/K |
| 8 | Canine | M44R/Q | C154G/R | R188L/N | R306P/N/A/V | E309N/L/T/S/K |
| 9 | Mole | M44R/Q | C154G/R | R188L/N | — | E309N/L/T/S/K |
| 10 | Pika | M44R/Q | C154G/R | R188L/N | R306P/N/A/V | E309N/L/T/S/K |
| 11 | Hedgehog | M44R/Q | C154G/R | R188L/N | R309P/N/A/V | E312N/L/T/S/K |
| 12 | Tree shrew | — | C154G/R | R188L/N | R306P/N/A/V | E309N/L/T/S/K |
| 13 | Platypus | M44R/Q | C163G/R | R197L/N | R319P/N/A/V | E322N/L/T/S/K |
| 14 | Canary | — | C153G/R | R187L/N | R309P/N/A/V | — |
| 15 | Neopelma | — | C154G/R | R188L/N | R310P/N/A/V | E311N/L/T/S/K |
| 16 | Alligator | — | — | R188L/N | R310P/N/A/V | E313N/L/T/S/K |
| 18 | Xenopus | — | — | R188L/N | R307P/N/A/V | E310N/L/T/S/K |
| 21 | Brown Trout | — | — | R188L/N | — | E310N/L/T/S/K |
| 23 | Electric eel | — | — | R188L/N | — | — |
| 25 | Walking fish | — | — | R188L/N | R305P/N/A/V | E308N/L/T/S/K |
| 27 | Guppy | — | — | R188L/N | R305P/N/A/V | E308N/L/T/S/K |
| 29 | Rat | — | — | R188L/N | R306P/N/A/V | E309N/L/T/S/K |
| 31 | Piliocolobus | — | — | R188L/N | R306P/N/A/V | E309N/L/T/S/K |
| 32 | Pig | M44R/Q | C154G/R | R188L/N | R306P/N/A/V | E309N/L/T/S/K |
| 35 | Water buffalo | M44R/Q | C154G/R | R188L/N | R305P/N/A/V | E308N/L/T/S/K |
| 36 | Marmot | M44R/Q | C154G/R | R188L/N | R306P/N/A/V | E309N/L/T/S/K |

In some embodiments, TdT variants of the invention, such as those described above, may comprise a peptide affinity tag for convenient purification after expression in a production process. In some embodiments such peptide affinity tags may be left on the TdT variant when used in template-free DNA synthesis, as typically neither N-terminal nor C-terminal peptide affinity tags do not affect activity. However, the additional peptides on either the N-terminus or C-terminus do increase the radius of gyration of the enzyme, thereby reducing its compactness and ability to penetrate interior spaces of a porous synthesis support. One aspect of the invention is the discovery that a peptide affinity tag could be located in the interior of a TdT variant by inserting it into the loop 2 region of the enzyme. Such interior peptide affinity tag may be inserted between any two successive amino acids of a loop 2 region, or they may be inserted by the exchange of one or more of the loop 2 amino acids. In some embodiments, a peptide affinity tag is inserted into a loop 2 region by exchanging it with a segment of amino acids in the loop 2 region of the same length (up to the length of the loop 2 region). In other embodiments, a peptide affinity tag may have a length exceeding that of the loop 2 region, so that the peptide affinity tag may be exchanged with the amino acids of the loop 2 region, thereby increasing the size of the loop 2 region. In an example of the latter embodiment, a 15-amino acid loop 2 region of SEQ ID NO: 2 may be exchanged for a 27-amino acid cellulose binding domain (Kimple, Curr. Protocols Protein Sci., 73: Unit 9.9 (2015), thereby increasing the size of the resulting loop 2 region. In some embodiments, the length of the loop 2 region will be unchanged by the insertion of a peptide affinity tag; in other embodiments, the length of the loop 2 region after insertion will be equal to or less than the native loop 2 region. That is, in some embodiments, a peptide affinity tag is selected that has a length which is less than or equal to the length of the loop 2 region being modified.

TABLE 2
Location of Loop 2 Regions in Exemplary TdT Variants
(determined by alignment with mouse TdT)

| SEQ ID | | |
|---|---|---|
| NO | Animal | Amino acid position |
| 1 | Mouse | 412-427 |
| 2 | Mouse | 264-279 |
| 3 | Bovine | 263-278 |
| 4 | Human | 263-278 |
| 5 | Chicken | 254-275 |
| 6 | Possum | 264-285 |
| 7 | Shrew | 264-279 |
| 8 | Canine | 264-279 |
| 9 | Mole | 264-279 |
| 10 | Pika | 264-279 |
| 11 | Hedgehog | 264-282 |
| 12 | Tree shrew | 264-279 |
| 13 | Platypus | 271-292 |
| 14 | Canary | 261-282 |
| 15 | Neopelma | 262-283 |
| 16 | Alligator | 262-283 |
| 18 | Xenopus | 262-280 |
| 21 | Brown Trout | 262-280 |
| 23 | Electric eel | 262-277 |
| 25 | Walking fish | 262-278 |
| 27 | Guppy | 262-278 |
| 29 | Rat | 263-279 |
| 31 | Piliocolobus | 264-279 |
| 32 | Pig | 264-279 |
| 35 | Water buffalo | 263-278 |
| 36 | Marmot | 264-279 |

TdT variants of the invention as described above each comprise an amino acid sequence having a percent sequence identity with a specified SEQ ID NO, subject to the presence of indicated substitutions. In some embodiments, the number and type of sequence differences between a TdT variant of the invention described in this manner and the specified SEQ ID NO may be due to substitutions, deletion and/or insertions, and the amino acids substituted, deleted and/or inserted may comprise any amino acid. In some embodiments, such deletions, substitutions and/or insertions comprise only naturally occurring amino acids. In some embodiments, substitutions comprise only conservative, or synonymous, amino acid changes, as described in Grantham, Science, 185: 862-864 (1974). That is, a substitution of an amino acid can occur only among members of its set of synonymous amino acids. In some embodiments, sets of synonymous amino acids that may be employed are set forth in Table 3A.

TABLE 3A
Synonymous Sets of Amino Acids I

| Amino Acid | Synonymous Set |
|---|---|
| Ser | Ser, Thr, Gly, Asn |
| Arg | Arg, Gln, Lys, Glu, His |
| Leu | Ile, Phe, Tyr, Met, Val, Leu |
| Pro | Gly, Ala, Thr, Pro |
| Thr | Pro, Ser, Ala, Gly, His, Gln, Thr |
| Ala | Gly, Thr, Pro, Ala |
| Val | Met, Tyr, Phe, Ile, Leu, Val |
| Gly | Gly, Ala, Thr, Pro, Ser |
| Ile | Met, Tyr, Phe, Val, Leu, Ile |
| Phe | Trp, Met, Tyr, Ile, Val, Leu, Phe |
| Tyr | Trp, Met, Phe, Ile, Val, Leu, Tyr |
| Cys | Cys, Ser, Thr |
| His | His, Glu, Lys, Gln, Thr, Arg |
| Gln | Gln, Glu, Lys, Asn, His, Thr, Arg |
| Asn | Asn, Gln, Asp, Ser |
| Lys | Lys, Glu, Gln, His, Arg |
| Asp | Asp, Glu, Asn |
| Glu | Glu, Asp, Lys, Asn, Gln, His, Arg |
| Met | Met, Phe, Ile, Val, Leu |
| Trp | Trp |

In some embodiments, sets of synonymous amino acids that may be employed are set forth in Table 3B.

TABLE 3B
Synonymous Sets of Amino Acids II

| Amino Acid | Synonymous Set |
|---|---|
| Ser | Ser |
| Arg | Arg, Lys, His |
| Leu | Ile, Phe, Met, Leu |
| Pro | Ala, Pro |
| Thr | Thr |
| Ala | Pro, Ala |
| Val | Met, Ile Val |
| Gly | Gly |
| Ile | Met, Phe, Val, Leu, Ile |
| Phe | Met, Tyr, Ile, Leu, Phe |
| Tyr | Trp, Met |
| Cys | Cys, Ser |
| His | His, Gln, Arg |
| Gln | Gln, Glu, His |
| Asn | Asn, Asp |
| Lys | Lys, Arg |
| Asp | Asp, Asn |
| Glu | Glu, Gln |
| Met | Met, Phe, Ile, Val, Leu |
| Trp | Trp |

### Measurement of Nucleotide Incorporation Activity

The efficiency of nucleotide incorporation by variants of the invention may be measured by an extension, or elongation, assay, e.g. as described in Boule et al (cited below); Bentolila et al (cited below); and Hiatt et al, U.S. Pat. No. 5,808,045, the latter of which is incorporated herein by reference. Briefly, in one form of such an assay, a fluorescently labeled oligonucleotide having a free 3′-hydroxyl is reacted under TdT extension conditions with a variant TdT to be tested for a predetermined duration in the presence of a reversibly blocked nucleoside triphosphate, after which the extension reaction is stopped and the amounts of extension products and unextended initiator oligonucleotide are quantified after separation by gel electrophoresis. By such assays, the incorporation efficiency of a variant TdT may be readily compared to the efficiencies of other variants or to that of wild type or reference TdTs, or other polymerases. In some embodiments, a measure of variant TdT efficiency may be a ratio (given as a percentage) of amount of extended product using the variant TdT over the amount of extended product using wild type TdT in an equivalent assay.

In some embodiments, the following particular extension assay may be used to measure incorporation efficiencies of TdTs: Primer used is the following:

(SEQ ID NO: 52)
5′-AAAAAAAAAAAAAAGGGG-3′

The primer has also an ATTO fluorescent dye on the 5′ extremity. Representative modified nucleotides used include 3′-O-amino-2′,3′-dideoxynucleotides-5′-triphosphates (ONH2, Firebird Biosciences), such as 3′-O-amino-2′,3′-dideoxyadenosine-5′-triphosphate. For each different variant tested, one tube is used for the reaction. The reagents are added in the tube, starting from water, and then in the order of Table 4. After 30 min at 37° C. the reaction is stopped by addition of formamide (Sigma).

TABLE 4
Extension Activity Assay Reagents

| | | |
|---|---|---|
| Reagent | Concentration | Volume |

| | | |
|---|---|---|
| H₂O | — | 12 μL |
| Activity buffer | 10x | 2 μL |

| | | | |
|---|---|---|---|
| dNTP | 250 | μM | 2 μL |
| Purified enzyme | 20 | μM | 2 μL |
| Fluorescent primer | 500 | nM | 2 μL |

The Activity buffer comprises, for example, TdT reaction buffer (available from New England Biolabs) supplemented with CoCl₂.

The product of the assay is analyzed by conventional polyacrylamide gel electrophoresis. For example, products of the above assay may be analyzed in a 16 percent polyacrylamide denaturing gel (Bio-Rad). Gels are made just before the analysis by pouring polyacrylamide inside glass plates and let it polymerize. The gel inside the glass plates is mounted on an adapted tank filed with TBE buffer (Sigma) for the electrophoresis step. The samples to be analyzed are loaded on the top of the gel. A voltage of 500 to 2,000V is applied between the top and bottom of the gel for 3 to 6 h at room temperature. After separation, gel fluorescence is scanned using, for example, a Typhoon scanner (GE Life Sciences). The gel image is analyzed using ImageJ software (imagej.nih.gov/ij/), or its equivalent, to calculate the percentage of incorporation of the modified nucleotides.

Hairpin completion assay. In one aspect, the invention includes methods of measuring the capability of a polymerase, such as a TdT variant, to incorporate a dNTP onto a 3′ end of a polynucleotide (i.e. a “test polynucleotide”). One such method comprises providing a test polynucleotide with a free 3′ hydroxyl under reaction conditions in which it is substantially only single stranded, but that upon extension with a polymerase, such as a TdT variant, forms a stable hairpin structure comprising a single stranded loop and a double stranded stem, thereby allowing detection of an extension of the 3′ end by the presence of the double stranded polynucleotide. The double stranded structure may be detected in a variety of ways including, but not limited to, fluorescent dyes that preferentially fluoresce upon intercalation into the double stranded structure, fluorescent resonance energy transfer (FRET) between an acceptor (or donor) on the extended polynucleotide and a donor (or acceptor) on an oligonucleotide that forms a triplex with the newly formed hairpin stem, FRET acceptors and donors that are both attached to the test polynucleotide and that are brought into FRET proximity upon formation of a hairpin, or the like. In some embodiments, a stem portion of a test polynucleotide after extension by a single nucleotide is in the range of 4 to 6 basepairs in length; in other embodiments, such stem portion is 4 to 5 basepairs in length; and in still other embodiments, such stem portion is 4 basepairs in length. In some embodiments, a test polynucleotide has a length in the range of from 10 to 20 nucleotides; in other embodiments, a test polynucleotide has a length in the range of from 12 to 15 nucleotides. In some embodiments, it is advantageous or convenient to extend the test polynucleotide with a nucleotide that maximizes the difference between the melting temperatures of the stem without extension and the stem with extension; thus, in some embodiments, a test polynucleotide is extended with a dC or dG (and accordingly the test polynucleotide is selected to have an appropriate complementary nucleotide for stem formation).

Exemplary test polynucleotides for hairpin completion assays include p875 (5′-CAGTTAAAAACT) (SEQ ID NO: 53) which is completed by extending with a dGTP; p876 (5′-GAGTTAAAACT) (SEQ ID NO: 54) which is completed by extending with a dCTP; and p877 (5′-CAGCAAGGCT) (SEQ ID NO: 55) which is completed by extending with a dGTP. Exemplary reaction conditions for such test polynucleotides may comprise: 2.5-5 μM of test polynucleotide, 1:4000 dilution of GelRed® (intercalating dye from Biotium, Inc., Fremont, CA), 200 mM Cacodylate KOH pH 6.8, 1 mM CoCl₂, 0-20% of DMSO and 3′-ONH2 dGTP and TdT at desired concentrations. Completion of the hairpin may be monitored by an increase in fluorescence of GelRed® dye using a conventional fluorimeter, such as a TECAN reader at a reaction temperature of 28-38° C., using an excitation filter set to 360 nm and an emission filter set to 635 nm. In some embodiments of this aspect of the invention, TdT variants may be tested for their capacity for template-free incorporate of nucleoside triphosphates by the following steps: (a) combining a test polynucleotide having a free 3′-hydroxyl, a TdT variant and a nucleoside triphosphate under conditions wherein the test polynucleotide is single stranded but upon incorporation of the nucleoside triphosphate forms a hairpin having a double stranded stem region, and (b) detecting the amount of double stranded stem regions formed as a measure of the capacity of the TdT variant to incorporate the nucleoside triphosphate. In some embodiments, the nucleoside triphosphate is a 3′-O-blocked nucleoside triphosphate.

Another test for coupling activity is referred to herein as the “tagct+A” assay. Similar reactions are carried out as with the above hairpin assays, except that the extension target is the short oligonucleotide, 5′-TAGCT, which forms a duplex with itself, thereby providing a double extended structure having greater relative stability (with 2 added nucleotides) than the extended hairpins (with a single extended nucleotide). One with ordinary skill would recognize that assays similar to the “tagct+A” assay may be constructed with different sequences for measuring coupling efficiency for different monomers.

Measurement of TdT Variant Stability

Increases in the stability of a protein may be measured in a variety of ways. Fluorescent-based thermal shift assays are particularly useful for such measurements because of their simplicity and low cost. Exemplary references describing thermal shift assays and their application to measuring protein stability are as follows: Pantoliano et al, J. Biomolecular Screening, 6(6): 429-440 (2001); Huynh et al, Curr. Protocol. Protein Science, 79: 28.9.1-28.9.14; Ericsson et al, Anal. Biochem., 357: 289-298 (2006); Niesen et al, Nature Protocols, 2(9): 2212-2221 (2007); and Pantoliano et al, U.S. Pat. No. 6,020,141, the latter of which is hereby incorporated by reference. The conceptual basis for such assays is that folded and unfolded proteins can be distinguished through exposure to a hydrophobic fluorescent probe. Such a probe is quenched in aqueous solution but will preferentially bind to the exposed hydrophobic interior of an unfolding protein leading to a sharp decrease in quenching so that a readily detected fluorescence emission can be studied as a function of temperature. Thermally induced unfolding is an irreversible unfolding process following a typical two-state model with a sharp transition between the folded and unfolded states, where melting temperature (Tm) is defined as the midpoint of temperature of the protein-unfolding transition. Melting temperatures obtained with such a so-called “thermofluor” method have been shown to correlate well for several proteins with values determined by other biophysical methods used for measuring protein stability, such as circular dichroism (CD), turbidity measurements, and differential scanning calorimetry.

Low fluorescence at room temperature indicates a well-folded protein. Fluorescence emission increases with increasing temperature, giving rise to a sigmoidal curve that represents cooperative unfolding of the protein. The resulting sigmoidal curves may be fit to a Boltzmann Equation to identify the melting temperature that occurs at the midpoint of the unfolding transition; alternatively, the Tm is easily identified by plotting the first derivative of the fluorescence emission as a function of temperature (−dF/dT), where the Tm corresponds to the minimum of the curve, Huynh et al (cited above).

A variety of fluorescent dyes may be used and are commercially available directly or as part of an assay kit. An exemplary fluorescent dye is SYPRO Orange. A conventional real-time PCR instrument may be used for temperature control and fluorescent detection. Typically a selection of buffers, salt concentrations and compositions, and other additives, e.g. DMSO, is made that corresponds to the incorporation or coupling conditions of a DNA synthesis reaction using a TdT variant. Exemplary coupling conditions are given below.

As mentioned above, in some embodiments, a stabilizing amino acid substitution (or equivalently, a stabilizing amino acid) at a specified position of an N-truncated TdT variant is an amino acid substitution which increases the melting temperature of said TdT variant by at least 1° C. relative to a melting temperature of a TdT of the same amino acid sequence except for the stabilizing amino acid substitution, where the melting temperature is determined by a fluorescence-based thermal shift assay. In some embodiments, such assay uses a SYPRO Orange dye. In still further embodiments, reaction conditions of such assay comprises coupling reaction conditions. In further embodiments, such stabilizing amino acid substitution increases the melting temperature of a TdT variant by 1.5° C.

In some embodiments, melting temperature is measured with a thermal shift assay in the following solution: 0.5 Cacodylate KOH buffer pH 7.4 with SYPRO Orange dye. Some routine optimization of dye and protein concentrations may be made for the assay, e.g. guidance for such optimization is provided in the above-cited references, especially Huynh et al (cited above).

Affinity Tags

A wide variety of peptide affinity tags may be inserted into the loop 2 region of TdT variants. Factors influencing selection of a peptide include, but are not limited to, (i) size of peptide affinity tag (peptides with a length of less than 20 amino acids are preferred), (ii) efficacy or yield of affinity purification, (iii) effect on coupling activity, (iv) effect on solubility and stability, (v) procedures for affinity capture and elution of a TdT, (vi) cost and scalability of purification procedure, and the like. Guidance for selecting a peptide affinity tag is described in the following references: Terpe, Appl. Microbiol. Biotechnol., 60: 523-533 (2003); Arnau et al, Protein Expression and Purification, 48: 1-13 (2006); Kimple et al, Curr. Protoc. Protein Sci., 73: Unit-9.9 (2015); Kimple et al, U.S. Pat. No. 7,309,575; Lichty et al, Protein Expression and Purification, 41: 98-105 (2005); and the like. Exemplary peptide affinity tags that may be used with the invention are listed in Table 5.

TABLE 5

| Name | Exemplary Peptide | Purification |
|---|---|---|
| Poly His | HHHHHH (H₂-H₁₀)* | IMAC |
| FLAG | DYKDDDDK | Antibody |
| FLAG short | DYKD | Antibody |
| c-myc | EQKLISEEDL | Antibody |
| Strep II | WSHPQFEK | Strep-Tactin |
| Poly Arg | RRRRR (R₅-R₆)* | Cation exchange resin |
| AU1 epitope | DTYRYI | Antibody |
| AU5 epitope | TDFYLK | Antibody |
| Bacteriophage T7 | MASMTGGQQMG | Antibody |
| epitope | | |
| Bacteriophage V5 | GKPIPNPLLGLDST | Antibody |
| epitope | | |
| Bluetongue virus | QYPALT | Antibody |
| tag | | |
| E2 epitope | SSTSSDFRDR | Antibody |
| EE-tag | EYMPME | Antibody |
| HSV epitope | KPPTPPPEPET | Antibody |
| Poly Asp | DDDDD | Anion exchange resin |
| Poly Phe | FFFFFFFFFFF | Phenyl-Sepharose/ethylene |
| | | glycol |
| Poly Cys | CCCC (C₄)* | Thiopropyl-sepharo sethiol- |
| | | containing reducing agent |
| S1 tag | NANNPDWDF | Antibody |
| S tag | KETAAAKFERQHMDS | S-fragment of RNase A |

*The subscript indicates the number of the indicated amino acid in the affinity tag.

In some embodiments, a peptide affinity tag is inserted into a loop 2 region of a TdT variant without removing any pre-existing amino acids from the loop, so that the size of the modified loop 2 is larger than the unmodified loop 2. In other embodiments, a peptide affinity tag is inserted into a loop 2 region and one or more pre-existing amino acids are removed. In still other embodiments, a segment of pre-existing amino acids having the same length as the peptide affinity tag are removed and replaced by the peptide affinity tag. In some embodiments, larger affinity tags, comprising protein fragments, may be inserted into the loop 2 region, such as, GFP, photoactive yellow protein, human influenza hemagglutinin, galactose-binding protein (GBP), HaloTag®, maltose-binding protein (MBP), PDZ domain, streptavadin-binding peptide (SBP), cellulose binding domain (CBP), and the like.

In some embodiments, the peptide affinity tags comprise poly-amino acid segments that do not require antibody binding for purification. Exemplary poly-amino acids include polyAsp, polyGlu, polyCys, polyPhe, polyAsp and polyHis. In some embodiment, said peptide affinity tag is H₂-H₁₀, particularly H₄-H₈. In a particular embodiment, the peptide affinity tag inserted into a loop 2 region is a 6-mer of histidine (H₆ or a segment “—HHHHHH—”).

In some embodiments of the invention, a TdT variant comprising a peptide affinity tag within its loop 2 region is used directly (i.e. without removal of the affinity tag) in a process of template-free enzymatic synthesis of oligonucleotides and polynucleotides, as described below. In particular, N-terminally truncated and stabilized TdT variants of the invention also comprising a peptide affinity tag within its loop 2 region may be used for template-free enzymatic synthesis of oligonucleotides and polynucleotides. For example, such synthesis may comprise the following steps: (a) providing an initiator having a 3′-terminal nucleotide having a free 3′-hydroxyl; (b) repeating cycles of the steps: (i) contacting under elongation conditions the initiator or elongated fragments having free 3′-O-hydroxyls with a 3′-O-blocked nucleoside triphosphate and a TdT variant, so that the initiator or elongated fragments are elongated by incorporation of a 3′-O-blocked nucleoside triphosphate to form 3′-O-blocked elongated fragments, and (ii) deblocking the elongated fragments to form elongated fragments having free 3′-hydroxyls, until the polynucleotide is formed; wherein the TdT variant has a loop 2 region comprising a peptide affinity tag inserted therein, and wherein the TdT variant (A) is capable of synthesizing a nucleic acid fragment without a template and (B) is capable of incorporating a 3′-O-protected-nucleotide onto a free 3′-hydroxyl of a polynucleotide.

Template-Free Enzymatic Synthesis of Oligonucleotides

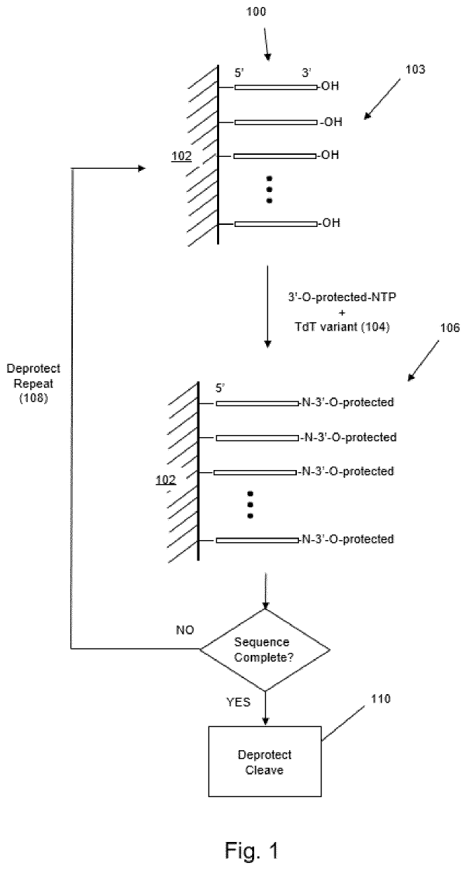

Generally, methods of template-free (or equivalently, “template-independent”) enzymatic DNA synthesis comprise repeated cycles of steps, such as are illustrated in FIG. 1, in which a predetermined nucleotide is coupled to an initiator or growing chain in each cycle. The general elements of template-free enzymatic synthesis is described in the following references: Ybert et al, International patent publication WO/2015/159023; Ybert et al, International patent publication WO/2017/216472; Hyman, U.S. Pat. No. 5,436,143; Hiatt et al, U.S. Pat. No. 5,763,594; Jensen et al, Biochemistry, 57: 1821-1832 (2018); Mathews et al, Organic & Biomolecular Chemistry, DOI: 0.1039/c6ob01371f (2016); Schmitz et al, Organic Lett., 1(11): 1729-1731 (1999).

Initiator polynucleotides (100) are provided, for example, attached to solid support (102), which have free 3′-hydroxyl groups (103). To the initiator polynucleotides (100) (or elongated initiator polynucleotides in subsequent cycles) are added a 3′-O-protected-dNTP and a template-free polymerase, such as a TdT or variant thereof (e.g. Ybert et al, WO/2017/216472; Champion et al, WO2019/135007) under conditions (140) effective for the enzymatic incorporation of the 3′-O-protected-dNTP onto the 3′ end of the initiator polynucleotides (100) (or elongated initiator polynucleotides). This reaction produces elongated initiator polynucleotides whose 3′-hydroxyls are protected (106). If the elongated sequence is not complete, then another cycle of addition is implemented (108). If the elongated initiator polynucleotide contains a competed sequence, then the 3′-O-protection group may be removed, or deprotected, and the desired sequence may be cleaved from the original initiator polynucleotide (110). Such cleavage may be carried out using any of a variety of single strand cleavage techniques, for example, by inserting a cleavable nucleotide at a predetermined location within the original initiator polynucleotide. An exemplary cleavable nucleotide may be a uracil nucleotide which is cleaved by uracil DNA glycosylase. If the elongated initiator polynucleotide does not contain a completed sequence, then the 3′-O-protection groups are removed to expose free 3′-hydroxyls (103) and the elongated initiator polynucleotides are subjected to another cycle of nucleotide addition and deprotection.

As used herein, the terms “protected” and “blocked” in reference to specified groups, such as, a 3′-hydroxyls of a nucleotide or a nucleoside, are used interchangeably and are intended to mean a moiety is attached covalently to the specified group that prevents a chemical change to the group during a chemical or enzymatic process. Whenever the specified group is a 3′-hydroxyl of a nucleoside triphosphate, or an extended fragment (or “extension intermediate”) in which a 3′-protected (or blocked)-nucleoside triphosphate has been incorporated, the prevented chemical change is a further, or subsequent, extension of the extended fragment (or “extension intermediate”) by an enzymatic coupling reaction.

As used herein, an “initiator” (or equivalent terms, such as, “initiating fragment,” “initiator nucleic acid,” “initiator oligonucleotide,” or the like) usually refers to a short oligonucleotide sequence with a free 3′-hydroxyl at its end, which can be further elongated by a template-free polymerase, such as TdT. In one embodiment, the initiating fragment is a DNA initiating fragment. In an alternative embodiment, the initiating fragment is an RNA initiating fragment. In some embodiments, an initiating fragment possesses between 3 and 100 nucleotides, in particular between 3 and 20 nucleotides. In some embodiments, the initiating fragment is single-stranded. In alternative embodiments, the initiating fragment may be double-stranded. In some embodiments, an initiator oligonucleotide may be attached to a synthesis support by its 5′end; and in other embodiments, an initiator oligonucleotide may be attached indirectly to a synthesis support by forming a duplex with a complementary oligonucleotide that is directly attached to the synthesis support, e.g. through a covalent bond. In some embodiments a synthesis support is a solid support which may be a discrete region of a solid planar solid, or may be a bead.

In some embodiments, an initiator may comprise a non-nucleic acid compound having a free hydroxyl to which a TdT may couple a 3′-O-protected dNTP, e.g. Baiga, U.S. patent publications US2019/0078065 and US2019/0078126.

After synthesis is completed polynucleotides with the desired nucleotide sequence may be released from initiators and the solid supports by cleavage. A wide variety of cleavable linkages or cleavable nucleotides may be used for this purpose. In some embodiments, cleaving the desired polynucleotide leaves a natural free 5′-hydroxyl on a cleaved strand; however, in alternative embodiments, a cleaving step may leave a moiety, e.g. a 5′-phosphate, that may be removed in a subsequent step, e.g. by phosphatase treatment. Cleaving steps may be carried out chemically, thermally, enzymatically or by photochemical methods. In some embodiments, cleavable nucleotides may be nucleotide analogs such as deoxyuridine or 8-oxo-deoxyguanosine that are recognized by specific glycosylases (e.g. uracil deoxyglycosylase followed by endonuclease VIII, and 8-oxoguanine DNA glycosylase, respectively). In some embodiments, cleavage may be accomplished by providing initiators with a deoxyinosine as the penultimate 3′ nucleotide, which may be cleaved by endonuclease V at the 3′ end of the initiator leaving a 5′-phosphate on the released polynucleotide. Further methods for cleaving single stranded polynucleotides are disclosed in the following references, which are incorporated by reference: U.S. Pat. Nos. 5,739,386, 5,700,642 and 5,830,655; and U.S. Patent Publication Nos. 2003/0186226 and 2004/0106728; and in Urdea and Horn, U.S. Pat. No. 5,367,066.

In some embodiments, cleavage by glycosylases and/or endonucleases may require a double stranded DNA substrate.

Returning to FIG. 1, in some embodiments, an ordered sequence of nucleotides are coupled to an initiator nucleic acid using a template-free polymerase, such as TdT, in the presence of 3′-O-protected dNTPs in each synthesis step. In some embodiments, the method of synthesizing an oligonucleotide comprises the steps of (a) providing an initiator (e.g. (100)) having a free 3′-hydroxyl (103); (b) reacting (104) under extension conditions the initiator or an extension intermediate having a free 3′-hydroxyl with a template-free polymerase in the presence of a 3′-O-protected nucleoside triphosphate to produce a 3′-O-protected extension intermediate (106); (c) deprotecting the extension intermediate to produce an extension intermediate with a free 3′-hydroxyl; and (d) repeating steps (b) and (c) (108) until the polynucleotide is synthesized (110). (Sometimes the terms “extension intermediate” and “elongation fragment” are used interchangeably).

In other words, the method of synthesizing the polynucleotide or oligonucleotide comprises (a) a step of providing an initiator (e.g.(100)) having a 3′-terminal nucleotide having a free 3′-hydroxyl (103), (b) a step of contacting (104) under elongation conditions the initiator having a free 3′-O-hydroxyls (103) with a 3′-O-blocked nucleoside triphosphate and a TdT variant according the invention, so that the initiator(e.g.:(100)) is elongated by incorporation of a 3′-O-blocked nucleoside triphosphate to form a 3′-O-blocked elongated fragment, and (c) a step of deblocking the elongated fragment (106) to form an elongated fragment having a free 3′-hydroxyl and (d) a step or repeating steps (b) and (c) (108) by contacting under elongation conditions the elongated fragment obtained in the step of deblocking (c), until the polynucleotide or oligonucleotide is formed (100).

In some embodiments, an initiator is provided as an oligonucleotide attached to a solid support, e.g. by its 5′ end. The above method may also include washing steps after the reaction, or extension, step, as well as after the de-protecting step. For example, the step of reacting may include a sub-step of removing unincorporated nucleoside triphosphates, e.g. by washing, after a predetermined incubation period, or reaction time. Such predetermined incubation periods or reaction times may be a few seconds, e.g. 30 sec, to several minutes, e.g. 30 min.

When the sequence of polynucleotides on a synthesis support includes reverse complementary subsequences, secondary intra-molecular or cross-molecular structures may be created by the formation of hydrogen bonds between the reverse complementary regions. In some embodiments, base protecting moieties for exocyclic amines are selected so that hydrogens of the protected nitrogen cannot participate in hydrogen bonding, thereby preventing the formation of such secondary structures. That is, base protecting moieties may be employed to prevent the formation of hydrogen bonds, such as are formed in normal base pairing, for example, between nucleosides A and T and between G and C. At the end of a synthesis, the base protecting moieties may be removed and the polynucleotide product may be cleaved from the solid support, for example, by cleaving it from its initiator.

In addition to providing 3′-O-blocked dNTP monomers with base protection groups, elongation reactions may be performed at higher temperatures using thermal stable template-free polymerases. For example, a thermal stable template-free polymerase having activity above 40° C. may be employed; or, in some embodiments, a thermal stable template-free polymerase having activity in the range of from 40-85° C. may be employed; or, in some embodiments, a thermal stable template-free polymerase having activity in the range of from 40-65° C. may be employed.

In some embodiments, elongation conditions may include adding solvents to an elongation reaction mixture that inhibit hydrogen bonding or base stacking. Such solvents include water miscible solvents with low dielectric constants, such as dimethyl sulfoxide (DMSO), methanol, and the like. Likewise, in some embodiments, elongation conditions may include the provision of chaotropic agents that include, but are not limited to, n-butanol, ethanol, guanidinium chloride, lithium perchlorate, lithium acetate, magnesium chloride, phenol, 2-propanol, sodium dodecyl sulfate, thiourea, urea, and the like. In some embodiments, elongation conditions include the presence of a secondary-structure-suppressing amount of DMSO. In some embodiments, elongation conditions may include the provision of DNA binding proteins that inhibit the formation of secondary structures, wherein such proteins include, but are not limited to, single-stranded binding proteins, helicases, DNA glycolases, and the like. 3′-O-blocked dNTPs without base protection may be purchased from commercial vendors or synthesized using published techniques, e.g. U.S. Pat. No. 7,057,026; Guo et al, Proc. Natl. Acad. Sci., 105(27): 9145-9150 (2008); Benner, U.S. Pat. Nos. 7,544,794 and 8,212,020; International patent publications WO2004/005667, WO91/06678; Canard et al, Gene (cited herein); Metzker et al, Nucleic Acids Research, 22: 4259-4267 (1994); Meng et al, J. Org. Chem., 14: 3248-3252 (3006); U.S. patent publication 2005/037991. 3′-O-blocked dNTPs with base protection may be synthesized as described below.

When base-protected dNTPs are employed the above method of FIG. 1 may further include a step (e) removing base protecting moieties, which in the case of acyl or amidine protection groups may (for example) include treating with concentrated ammonia.

The above method may also include capping step(s) as well as washing steps after the reacting, or extending, step, as well as after the deprotecting step. As mentioned above, in some embodiments, capping steps may be included in which non-extended free 3′-hydroxyls are reacted with compounds that prevents any further extensions of the capped strand. In some embodiments, such compound may be a dideoxynucleoside triphosphate. In other embodiments, non-extended strands with free 3′-hydroxyls may be degraded by treating them with a 3′-exonuclease activity, e.g. Exo I. For example, see Hyman, U.S. Pat. No. 5,436,143. Likewise, in some embodiments, strands that fail to be deblocked may be treated to either remove the strand or render it inert to further extensions.

In some embodiments, reaction conditions for an elongation step (also sometimes referred to as an extension step or a coupling step) may comprising the following: 2.0 μM purified TdT; 125-600 μM 3′-O-blocked dNTP (e.g. 3′-O—NH₂-blocked dNTP); about 10 to about 500 mM potassium cacodylate buffer (pH between 6.5 and 7.5) and from about 0.01 to about 10 mM of a divalent cation (e.g. CoCl₂ or MnCl₂), where the elongation reaction may be carried out in a 50 μL reaction volume, at a temperature within the range RT to 45° C., for 3 minutes. In embodiments, in which the 3′-O-blocked dNTPs are 3′-O—NH₂-blocked dNTPs, reaction conditions for a deblocking step may comprise the following: 700 mM NaNO₂; 1 M sodium acetate (adjusted with acetic acid to pH in the range of 4.8-6.5), where the deblocking reaction may be carried out in a 50 μL volume, at a temperature within the range of RT to 45° C. for 30 seconds to several minutes. Washes may be performed with the cacodylate buffer without the components of the coupling reaction (e.g. enzyme, monomer, divalent cations).

Depending on particular applications, the steps of deblocking and/or cleaving may include a variety of chemical or physical conditions, e.g. light, heat, pH, presence of specific reagents, such as enzymes, which are able to cleave a specified chemical bond. Guidance in selecting 3′-O-blocking groups and corresponding de-blocking conditions may be found in the following references, which are incorporated by reference: Benner, U.S. patents 7544794 and 8212020; U.S. Pat. Nos. 5,808,045; 8,808,988; International patent publication WO91/06678; and references cited below. In some embodiments, the cleaving agent (also sometimes referred to as a de-blocking reagent or agent) is a chemical cleaving agent, such as, for example, dithiothreitol (DTT). In alternative embodiments, a cleaving agent may be an enzymatic cleaving agent, such as, for example, a phosphatase, which may cleave a 3′-phosphate blocking group. It will be understood by the person skilled in the art that the selection of deblocking agent depends on the type of 3′-nucleotide blocking group used, whether one or multiple blocking groups are being used, whether initiators are attached to living cells or organisms or to solid supports, and the like, that necessitate mild treatment. For example, a phosphine, such as tris(2-carboxyethyl)phosphine (TCEP) can be used to cleave a 3′O-azidomethyl groups, palladium complexes can be used to cleave a 3′O-allyl groups, or sodium nitrite can be used to cleave a 3′O-amino group. In particular embodiments, the cleaving reaction involves TCEP, a palladium complex or sodium nitrite.

As noted above, in some embodiments it is desirable to employ two or more blocking groups that may be removed using orthogonal de-blocking conditions. The following exemplary pairs of blocking groups may be used in parallel synthesis embodiments. It is understood that other blocking group pairs, or groups containing more than two, may be available for use in these embodiments of the invention.

| | |
|---|---|
| 3′-O—NH2 | 3′-O-azidomethyl |
| 3′-O—NH2 | 3′-O-allyl |
| 3′-O—NH2 | 3′-O-phosphate |
| 3′-O-azidomethyl | 3′-O-allyl |
| 3′-O-azidomethyl | 3′-O-phosphate |
| 3′-O-allyl | 3′-O-phosphate |

Synthesizing oligonucleotides on living cells requires mild deblocking, or deprotection, conditions, that is, conditions that do not disrupt cellular membranes, denature proteins, interfere with key cellular functions, or the like. In some embodiments, deprotection conditions are within a range of physiological conditions compatible with cell survival. In such embodiments, enzymatic deprotection is desirable because it may be carried out under physiological conditions. In some embodiments specific enzymatically removable blocking groups are associated with specific enzymes for their removal. For example, ester- or acyl-based blocking groups may be removed with an esterase, such as acetylesterase, or like enzyme, and a phosphate blocking group may be removed with a 3′ phosphatase, such as T4 polynucleotide kinase. By way of example, 3′-O-phosphates may be removed by treatment with as solution of 100 mM Tris-HCl (pH 6.5) 10 mM MgCl₂, 5 mM 2-mercaptoethanol, and one Unit T4 polynucleotide kinase. The reaction proceeds for one minute at a temperature of 37° C.

A “3′-phosphate-blocked” or “3′-phosphate-protected” nucleotide refers to nucleotides in which the hydroxyl group at the 3′-position is blocked by the presence of a phosphate containing moiety. Examples of 3′-phosphate-blocked nucleotides in accordance with the invention are nucleotidyl-3′-phosphate monoester/nucleotidyl-2′,3′-cyclic phosphate, nucicotidyl-2′-phosphate monoester and nucleotidyl-2′ or 3′-alkylphosphate diester, and nucleotidyl-2′ or 3′-pyrophosphate. Thiophosphate or other analogs of such compounds can also be used, provided that the substitution does not prevent dephosphorylation resulting in a free 3′-OH by a phosphatase.

Further examples of synthesis and enzymatic deprotection of 3′-O-ester-protected dNTPs or 3′-O-phosphate-protected dNTPs are described in the following references: Canard et al, Proc. Natl. Acad. Sci., 92:10859-10863 (1995); Canard et al, Gene, 148: 1-6 (1994); Cameron et al, Biochemistry, 16(23): 5120-5126 (1977); Rasolonjatovo et al, Nucleosides & Nucleotides, 18(4&5): 1021-1022 (1999); Ferrero et al, Monatshefte fur Chemie, 131: 585-616 (2000); Taunton-Rigby et al, J. Org. Chem., 38(5): 977-985 (1973); Uemura et al, Tetrahedron Lett., 30(29): 3819-3820 (1989); Becker et al, J. Biol. Chem., 242(5): 936-950 (1967); Tsien, International patent publication WO1991/006678.

In some embodiments, the modified nucleotides comprise a modified nucleotide or nucleoside molecule comprising a purine or pyrimidine base and a ribose or deoxyribose sugar moiety having a removable 3′-OH blocking group covalently attached thereto, such that the 3′ carbon atom has attached a group of the structure:

—O—Z

wherein —Z is any of —C(R′)₂—O—R″, —C(R′)₂—N(R″)₂, —C(R′)₂—N(H)R″, —C(R′)₂—S—R″ and —C(R′)₂—F, wherein each R″ is or is part of a removable protecting group; each R′ is independently a hydrogen atom, an alkyl, substituted alkyl, arylalkyl, alkenyl, alkynyl, aryl, heteroaryl, heterocyclic, acyl, cyano, alkoxy, aryloxy, heteroaryloxy or amido group, or a detectable label attached through a linking group; with the proviso that in some embodiments such substituents have up to 10 carbon atoms and/or up to 5 oxygen or nitrogen heteroatoms; or (R′)₂ represents a group of formula ═C(R′″)₂ wherein each R′″ may be the same or different and is selected from the group comprising hydrogen and halogen atoms and alkyl groups, with the proviso that in some embodiments the alkyl of each R′″ has from 1 to 3 carbon atoms; and wherein the molecule may be reacted to yield an intermediate in which each R″ is exchanged for H or, where Z is —(R′)₂—F, the F is exchanged for OH, SH or NH₂, preferably OH, which intermediate dissociates under aqueous conditions to afford a molecule with a free 3′-OH; with the proviso that where Z is —C(R′)₂—S—R″, both R′ groups are not H. In certain embodiments, R′ of the modified nucleotide or nucleoside is an alkyl or substituted alkyl, with the proviso that such alkyl or substituted alkyl has from 1 to 10 carbon atoms and from 0 to 4 oxygen or nitrogen heteroatoms. In certain embodiments, —Z of the modified nucleotide or nucleoside is of formula —C(R′)₂—N3. In certain embodiments, Z is an azidomethyl group.

In some embodiments, Z is a cleavable organic moiety with or without heteroatoms having a molecular weight of 200 or less. In other embodiments, Z is a cleavable organic moiety with or without heteroatoms having a molecular weight of 100 or less. In other embodiments, Z is a cleavable organic moiety with or without heteroatoms having a molecular weight of 50 or less. In some embodiments, Z is an enzymatically cleavable organic moiety with or without heteroatoms having a molecular weight of 200 or less. In other embodiments, Z is an enzymatically cleavable organic moiety with or without heteroatoms having a molecular weight of 100 or less. In other embodiments, Z is an enzymatically cleavable organic moiety with or without heteroatoms having a molecular weight of 50 or less. In other embodiments, Z is an enzymatically cleavable ester group having a molecular weight of 200 or less. In other embodiments, Z is a phosphate group removable by a 3′-phosphatase. In some embodiments, one or more of the following 3′-phosphatases may be used with the manufacturer's recommended protocols: T4 polynucleotide kinase, calf intestinal alkaline phosphatase, recombinant shrimp alkaline phosphatase (e.g. available from New England Biolabs, Beverly, MA)

In a further embodiments, the 3′-blocked nucleotide triphosphate is blocked by either a 3′-O-azidomethyl, 3′-O—NH₂ or 3′-O-allyl group. In other embodiments, 3′-O-blocking groups of the invention include 3′-O-methyl, 3′-O-(2-nitrobenzyl), 3′-O-allyl, 3′-O-amine, 3′-O-azidomethyl, 3′-O-tert-butoxy ethoxy, 3′-O-(2-cyanoethyl), 3′-O-nitro, and 3′-O-propargyl. In other embodiments, the 3′-blocked nucleotide triphosphate is blocked by either a 3′-O-azidomethyl or a 3′-O—NH2. Synthesis and use of such 3′-blocked nucleoside triphosphates are disclosed in the following references: U.S. Pat. Nos. 9,410,197; 8,808,988; 6,664,097; 5,744,595; 7,544,794; 8,034,923; 8,212,020; 10472383; Guo et al, Proc. Natl. Acad. Sci., 105(27): 9145-9150 (2008); and like references.

In some embodiments, 3′-O— protection groups are electrochemically labile groups. That is, deprotection or cleavage of the protection group is accomplished by changing the electrochemical conditions in the vicinity of the protection group which result in cleavage. Such changes in electrochemical conditions may be brought about by changing or applying a physical quantity, such as a voltage difference or light to activate auxiliary species which, in turn, cause changes in the electrochemical conditions at the site of the protection group, such as an increase or decrease in pH. In some embodiments, electrochemically labile groups include, for example, pH-sensitive protection groups that are cleaved whenever the pH is changed to a predetermined value. In other embodiments, electrochemically labile groups include protecting groups which are cleaved directly whenever reducing or oxidizing conditions are changed, for example, by increasing or decreasing a voltage difference at the site of the protection group.

Production of Variant TdTs

Variants of the invention may be produced by mutating known reference or wild type TdT-coding polynucleotides, then expressing it using conventional molecular biology techniques. For example, a desired gene or DNA fragment encoding a polypeptide of desired sequence may be assembled from synthetic fragments using conventional molecular biology techniques, e.g. using protocols described by Stemmer et al, Gene, 164: 49-53 (1995); Kodumal et al, Proc. Natl. Acad. Sci., 101: 15573-15578 (2004); or the like, or such gene or DNA fragment may be directly cloned from cells of a selected species using conventional protocols, e.g. described by Boule et al, Mol. Biotechnology, 10: 199-208 (1998), or Bentolila et al, EMBO J., 14: 4221-4229 (1995); or the like.

An isolated gene encoding a desired TdT variant may be inserted into an expression vector, such as pET32 (Novagen) to give an expression vector which then may be used to make and express variant TdT proteins using conventional protocols. Vectors with the correct sequence may be transformed in E. coli producer strains.

Transformed strains are cultured using conventional techniques to pellets from which TdT protein is extracted. For example, previously prepared pellets are thawed in 30 to 37° C. water bath. Once fully thawed, pellets are resuspended in lysis buffer composed of 50 mM tris-HCL (Sigma) pH 7.5, 150 mM NaCl (Sigma), 0.5 mM mercaptoethanol (Sigma), 5% glycerol (Sigma), 20 mM imidazole (Sigma) and 1 tab for 100 mL of protease cocktail inhibitor (Thermofisher). Careful resuspension is carried out in order to avoid premature lysis and remaining of aggregates. Resuspended cells are lysed through several cycles of French press, until full color homogeneity is obtained. Usual pressure used is 14,000 psi. Lysate is then centrifuged for 1 h to 1 h30 at 10,000 rpm. Centrifugate is pass through a 0.2 μm filter to remove any debris before column purification.

TdT protein may be purified from the centrifugate in a one-step affinity procedure. For example, Ni-NTA affinity column (GE Healthcare) may be used to bind the TdT polymerases. Initially the column is washed and equilibrated with 15 column volumes of 50 mM tris-HCL (Sigma) pH 7.5, 150 mM NaCl (Sigma) and 20 mM imidazole (Sigma). TdT polymerases are bound to the column after equilibration; then, a washing buffer, for example, composed of 50 mM tris-HCL (Sigma) pH 7.5, 500 mM NaCl (Sigma) and 20 mM imidazole (Sigma), may be applied to the column for 15 column volumes. After such washing, the TdT polymerases are eluted with 50 mM tris-HCL (Sigma) pH 7.5, 500 mM NaCl (Sigma) and 0.5M imidazole (Sigma). Fractions corresponding to the highest concentration of TdT polymerases of interest are collected and pooled in a single sample. The pooled fractions are dialyzed against the dialysis buffer (20 mM Tris-HCl, pH 6.8, 200 mM Na Cl, 50 mM MgOAc, 100 mM [NH4]2SO4). The dialysate is subsequently concentrated with the help of concentration filters (Amicon Ultra-30, Merk Millipore). Concentrated enzyme is distributed in small aliquots, 50% glycerol final is added, and those aliquots are then frozen at −20° C. and stored for long term. 5 μL of various fraction of the purified enzymes are analyzed in SDSPAGE gels.

In some embodiments, a TdT variant may be operably linked to a linker moiety including a covalent or non-covalent bond; amino acid tag (e.g., poly-amino acid tag, poly-His tag, 6His-tag, or the like); chemical compound (e.g., polyethylene glycol); protein-protein binding pair (e.g., biotin-avidin); affinity coupling; capture probes; or any combination of these. The linker moiety can be separate from or part of a TdT variant. An exemplary His-tag for use with TdT variants of the invention is MASSHHHHHHSSGSENLYFQTGSSG- (SEQ ID NO: 56)). The tag-linker moiety does not interfere with the nucleotide binding activity, or catalytic activity of the TdT variant.

Affinity purification using poly His and other poly-amino acid tags is well-known in the art. Exemplary references include Malhotra, “Tagging for protein expression,” chapter 16, Methods in Enzymology, 463: 239-258 (2009); Loughran et al, “Purification of poly-histidine-tagged proteins,” Methods in Molecular Biology, 681: 311-335 (2011); Loughran et al, “Purification of polyhistidine-tagged protiens,” Methods in Molecular Biology, 1485: 275-303 (2017); and the like. Briefly, in some embodiments, proteins incorporating a polyhistidine tag may be purified using immobilized metal affinity chromatography (IMAC). For example, a nitrilotriacetic acid (NTA) resin and nickel ion (Ni⁺²) may be used for binding, after which the protein may be eluted with imidazole in a phosphate or Tris buffer, pH 8.

In some embodiments, TdT variants of the invention include an affinity tag. For example, SEQ ID NO: 43 (M77) is the same as SEQ ID NO: 42 with a His tag conjugated to its N-terminus; SEQ ID NO: 45 (C1-40) is the same as SEQ ID NO: 44 with a His tag conjugated to its N-terminus; SEQ ID NO: 47 (A2-20) is the same as SEQ ID NO: 46 with a His tag conjugated to its N-terminus; SEQ ID NO: 49 (ii-ell-40) is the same as SEQ ID NO: 48 with a His tag conjugated to its N-terminus; and SEQ ID NO: 51 (9-40) is the same as SEQ ID NO: 50 with a His tag conjugated to its N-terminus. Each of the amino acid sequences of the foregoing SEQ ID NOs are TdT variants of the invention.

In additional embodiments, the manufacture of TdT variants of the invention may include the formation of fusions between a peptide or protein fragment and the N-terminus or C-terminus of the variant, wherein the peptide or protein fragment is used for affinity purification. After purification of the fusion protein the peptide or protein fragment may be cleaved prior to using the TdT variant for enzymatic polynucleotide synthesis, or in some embodiments, the fusion protein may be used without cleavage of the peptide or protein fragment. Exemplary peptides and protein fragments for such fusion proteins include, but are not limited to, green fluorescent protein (GFP), photoactive yellow protein, human influenza hemagglutinin, galactose-binding protein (GBP), HaloTag®, maltose-binding protein (MBP), PDZ domain, streptavadin-binding peptide (SBP), cellulose binding domain (CBP), and the like, e.g. as described in Kemple et al, Current Protocols in Protein Science (cited above).

The above processes, or equivalent processes, result in an isolated TdT variant that may be mixed with a variety of reagents, such as, salts, pH buffers, carrier compounds, and the like, that are necessary or useful for activity and/or preservation.

Kits for Practicing Methods of the Invention

The invention includes a variety of kits for practicing methods of the invention. In one aspect, kits of the invention comprise a TdT variant of the invention in a formulation suitable for carrying out template-free enzymatic polynucleotide synthesis as described herein. Such kits may also include synthesis buffers that provide reaction conditions for optimizing the template-free addition or incorporation of a 3′-O-protected dNTP to a growing strand. In some embodiments, kits of the invention further include 3′-O-reversibly protected dNTPs. In such embodiments, the 3′-O-reversibly protected dNTPs may comprise 3′-O-amino-dNTPs or 3′-O-azidomethyl-dNTPs. In further embodiments, kits may include one or more of the following items, either separately or together with the above-mentioned items: (i) deprotection reagents for carrying out a deprotecting step as described herein, (ii) solid supports with initiators attached thereto, (iii) cleavage reagents for releasing completed polynucleotides from solid supports, (iv) wash reagents or buffers for removing unreacted 3′-O-protected dNTPs at the end of an enzymatic addition or coupling step, and (v) post-synthesis processing reagents, such as purification columns, desalting reagents, eluting reagents, and the like. In some embodiments, kits of the invention may include arrays of reaction wells for carrying out multiple synthesis reactions in a single operation. In some embodiments, such arrays may be conventional filter plates comprising 24-, 48-, 96-, 384- or 1536-wells.

In regard to items (ii) and (iii) above, certain initiators and cleavage reagents go together. For example, an initiator comprising an inosine cleavable nucleotide may come with an endonuclease V cleavage reagent; an initiator comprising a nitrobenzyl photocleavable linker may come with a suitable light source for cleaving the photocleavable linker; an initiator comprising a uracil may come with a uracil DNA glycosylase cleavage reagent; and the like.

Exemplary TdT Variants

With Internal his Affinity Tags

N-terminally truncated TdT variants (45-751 and 46-737) with internal H₆ internal affinity tags were prepared having amino acid sequences SEQ ID NO: 57 and SEQ ID NO: 58, respectively. TdT variant 45-751 had a stabilizing Q8E mutation and a H₆ affinity tag exchanged with amino acid segment 275-280 and 46-737 had a stabilizing Q12E mutation and a H₆ affinity tag exchanged with amino acid segment 279-284. TdT variants M57 (SEQ ID NO: 38 with N-terminal His tag and linker SEQ ID NO: 56) and 46-737 were both tested (A) for stability using the termal stability assay described above, and (B) for coupling activity in a “tagct+A” assay as described above. TdT variant 46-737 was found to have slightly greater stability than that of M57 (melting temperature of 59.6° C. versus 58.5° C. for M57) and slightly less (80%) of the measured coupling activity of M57.

### Definitions

Amino acids are represented by either their one-letter or three-letters code according to the following nomenclature: A: alanine (Ala); C: cysteine (Cys); D: aspartic acid (Asp); E: glutamic acid (Glu); F: phenylalanine (Phe); G: glycine (Gly); H: histidine (His); I: isoleucine (Ile); K: lysine (Lys); L: leucine (Leu); M: methionine (Met); N: asparagine (Asn); P: proline (Pro); Q: glutamine (Gln); R: arginine (Arg); S: serine (Ser); T: threonine (Thr); V: valine (Val); W: tryptophan (Trp) and Y: tyrosine (Tyr).

“Functionally equivalent” in reference to a substituted residue means the substituted residue of a variant TdT has an identical functional role as a residue in a sequence of another TdT having a sequence homologous to SEQ ID NO: 1. Functionally equivalent residues may be identified by using sequence alignments, for example, using the Mutalin line alignment software (http://multalin.toulouse.inra.fr/multalin/multalin.html; 1988, Nucl. Acids Res., 16 (22), 25 10881-10890). After alignment, the functionally equivalent residues are at homologous positions on the different sequences considered. Sequence alignments and identification of functionally equivalent residues may be determined between any TdT and their natural variants, including inter-species.

“Isolated” in reference to a protein means such a compound which has been identified and separated and/or recovered from a component of its natural environment or from a heterogeneous reaction mixture. Contaminant components of a natural environment or reaction mixture are materials which would interfere with a protein's function, and may include enzymes, hormones, and other proteinaceous or nonproteinaceous solutes. In some embodiments, a protein of the invention is purified (1) to greater than 95% by weight of protein as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. When manufactured by recombinant methodologies, an isolated protein of the invention may include the protein of the invention in situ within recombinant cells since at least one component of the protein's natural environment will not be present. Ordinarily, an isolated protein of the invention is prepared by at least one purification step.

“Kit” refers to any delivery system for delivering materials or reagents for carrying out a method of the invention. In the context of reaction assays, such delivery systems include systems and/or compounds (such as dilutants, surfactants, carriers, or the like) that allow for the storage, transport, or delivery of reaction reagents (e.g., one or more TdT variants, reaction buffers, 3′-O-protected-dNTPs, deprotection reagents, solid suppprts with initiators attached, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. Such contents may be delivered to the intended recipient together or separately. For example, a first container may contain one or more TdT variants for use in a synthesis method, while a second or additional containers may contain deprotection agents, solid supports with initiators, 3′-O-protected dNTPs, or the like.

“Mutant” or “variant,” which are used interchangeably, refer to polypeptides derived from a natural or reference TdT polypeptide described herein, and comprising a modification or an alteration, i.e., a substitution, insertion, and/or deletion, at one or more positions. Variants may be obtained by various techniques well known in the art. In particular, examples of techniques for altering the DNA sequence encoding the wild-type protein, include, but are not limited to, site-directed mutagenesis, random mutagenesis, sequence shuffling and synthetic oligonucleotide construction. Mutagenesis activities consist in deleting, inserting or substituting one or several amino-acids in the sequence of a protein or in the case of the invention of a polymerase. The following terminology is used to designate a substitution: L238A denotes that amino acid residue (Leucine, L) at position 238 of a reference, or wild type, sequence is changed to an Alanine (A). A132V/I/M denotes that amino acid residue (Alanine, A) at position 132 of the parent sequence is substituted by one of the following amino acids: Valine (V), Isoleucine (I), or Methionine (M). The substitution can be a conservative or non-conservative substitution. Examples of conservative substitutions are within the groups of basic amino acids (arginine, lysine and histidine), acidic amino acids (glutamic acid and aspartic acid), polar amino acids (glutamine, asparagine and threonine), hydrophobic amino acids (methionine, leucine, isoleucine, cysteine and valine), aromatic amino acids (phenylalanine, tryptophan and tyrosine), and small amino acids (glycine, alanine and serine).

“Sequence identity” refers to the number (or fraction, usually expressed as a percentage) of matches (e.g., identical amino acid residues) between two sequences, such as two polypeptide sequences or two polynucleotide sequences. The sequence identity is determined by comparing the sequences when aligned so as to maximize overlap and identity while minimizing sequence gaps. In particular, sequence identity may be determined using any of a number of mathematical global or local alignment algorithms, depending on the length of the two sequences. Sequences of similar lengths are preferably aligned using a global alignment algorithm (e.g. Needleman and Wunsch algorithm; Needleman and Wunsch, 1970) which aligns the sequences optimally over the entire length, while sequences of substantially different lengths are preferably aligned using a local alignment algorithm (e.g. Smith and Waterman algorithm (Smith and Waterman, 1981) or Altschul algorithm (Altschul et al., 1997; Altschul et al., 2005)). Alignment for purposes of determining percent amino acid sequence identity can be achieved in various ways that are within the skill in the art, for instance, using publicly available computer software available on internet web sites such as http://blast.ncbi.nlm.nih.gov/ or ttp://www.ebi.ac.uk/Tools/emboss/. Those skilled in the art can determine appropriate parameters for measuring alignment, including any algorithm needed to achieve maximal alignment over the full length of the sequences being compared. For purposes herein, % amino acid sequence identity values refer to values generated using the pair wise sequence alignment program EMBOSS Needle, that creates an optimal global alignment of two sequences using the Needleman-Wunsch algorithm, wherein all search parameters are set to default values, i.e. Scoring matrix=BLOSUM62, Gap open=10, Gap extend=0.5, End gap penalty=false, End gap open=10 and End gap extend=0.5.

“Polynucleotide” or “oligonucleotide” are used interchangeably and each mean a linear polymer of nucleotide monomers or analogs thereof. Monomers making up polynucleotides and oligonucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like. Such monomers and their internucleosidic linkages may be naturally occurring or may be analogs thereof, e.g. naturally occurring or non-naturally occurring analogs. Non-naturally occurring analogs may include PNAs, phosphorothioate internucleosidic linkages, bases containing linking groups permitting the attachment of labels, such as fluorophores, or haptens, and the like. Whenever the use of an oligonucleotide or polynucleotide requires enzymatic processing, such as extension by a polymerase, ligation by a ligase, or the like, one of ordinary skill would understand that oligonucleotides or polynucleotides in those instances would not contain certain analogs of internucleosidic linkages, sugar moieties, or bases at any or some positions. Polynucleotides typically range in size from a few monomeric units, e.g. 5-40, when they are usually referred to as “oligonucleotides,” to several thousand monomeric units. Whenever a polynucleotide or oligonucleotide is represented by a sequence of letters (upper or lower case), such as “ATGCCTG,” it will be understood that the nucleotides are in 5′->3′ order from left to right and that “A” denotes deoxyadenosine, “C” denotes deoxycytidine, “G” denotes deoxyguanosine, and “T” denotes thymidine, “I” denotes deoxyinosine, “U” denotes uridine, unless otherwise indicated or obvious from context. Unless otherwise noted the terminology and atom numbering conventions will follow those disclosed in Strachan and Read, Human Molecular Genetics 2 (Wiley-Liss, New York, 1999). Usually polynucleotides comprise the four natural nucleosides (e.g. deoxyadenosine, deoxycytidine, deoxyguanosine, deoxythymidine for DNA or their ribose counterparts for RNA) linked by phosphodiester linkages; however, they may also comprise non-natural nucleotide analogs, e.g. including modified bases, sugars, or internucleosidic linkages. It is clear to those skilled in the art that where an enzyme has specific oligonucleotide or polynucleotide substrate requirements for activity, e.g. single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al, Molecular Cloning, Second Edition (Cold Spring Harbor Laboratory, New York, 1989), and like references. Likewise, the oligonucleotide and polynucleotide may refer to either a single stranded form or a double stranded form (i.e. duplexes of an oligonucleotide or polynucleotide and its respective complement). It will be clear to one of ordinary skill which form or whether both forms are intended from the context of the terms usage.

“Primer” means an oligonucleotide, either natural or synthetic that is capable, upon forming a duplex with a polynucleotide template, of acting as a point of initiation of nucleic acid synthesis and being extended from its 3′ end along the template so that an extended duplex is formed. Extension of a primer is usually carried out with a nucleic acid polymerase, such as a DNA or RNA polymerase. The sequence of nucleotides added in the extension process is determined by the sequence of the template polynucleotide. Usually primers are extended by a DNA polymerase. Primers usually have a length in the range of from 14 to 40 nucleotides, or in the range of from 18 to 36 nucleotides. Primers are employed in a variety of nucleic amplification reactions, for example, linear amplification reactions using a single primer, or polymerase chain reactions, employing two or more primers. Guidance for selecting the lengths and sequences of primers for particular applications is well known to those of ordinary skill in the art, as evidenced by the following references that are incorporated by reference: Dieffenbach, editor, PCR Primer: A Laboratory Manual, 2nd Edition (Cold Spring Harbor Press, New York, 2003).

A “substitution” means that an amino acid residue is replaced by another amino acid residue. Preferably, the term “substitution” refers to the replacement of an amino acid residue by another selected from the naturally-occurring standard 20 amino acid residues, rare naturally occurring amino acid residues (e.g. hydroxyproline, hydroxylysine, allohydroxylysine, 6-N-methylysine, N-ethylglycine, N-methylglycine, N-ethylasparagine, allo-isoleucine, N-methylisoleucine, N-methylvaline, pyroglutamine, aminobutyric acid, ornithine, norleucine, norvaline), and non-naturally occurring amino acid residue, often made synthetically, (e.g. cyclohexyl-alanine). Preferably, the term “substitution” refers to the replacement of an amino acid residue by another selected from the naturally-occurring standard 20 amino acid residues. The sign “+” indicates a combination of substitutions.

This disclosure is not intended to be limited to the scope of the particular forms set forth, but is intended to cover alternatives, modifications, and equivalents of the variations described herein. Further, the scope of the disclosure fully encompasses other variations that may become obvious to those skilled in the art in view of this disclosure. The scope of the present invention is limited only by the appended claims.

1. A terminal deoxynucleotidyl transferase (TdT) variant comprising an amino acid sequence at least ninety percent identical to an amino acid sequence selected from SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41, wherein a glutamine at position 4 of such amino acid sequence is substituted with a stabilizing amino acid; and wherein the TdT variant (i) is capable of synthesizing a nucleic acid fragment without a template and (ii) is capable of incorporating a 3′-O-protected-nucleotide onto a free 3′-hydroxyl of a polynucleotide.

2. The TdT variant according to claim 1, wherein said stabilizing amino acid is selected from the group consisting of E, S, D and N.

3. The TdT variant according to claim 1, wherein said stabilizing amino acid increases the melting temperature of said TdT variant by at least 1° C. relative to a melting temperature of a TdT of the same amino acid sequence except for said stabilizing amino acid substitution, wherein the melting temperature is determined by a fluorescence-based thermal shift assay.

4. The TdT variant according to claim 3, wherein said fluorescence-based thermal shift assay comprises SYPRO Orange dye in a solution of 0.5 cacodylate KOH buffer pH 7.4.

5. The TdT variant according to claim 1, having said amino acid sequence at least ninety percent identical to an amino acid sequence selected from SEQ ID NO: 17, 19, 20, 22, 24, 26, 28, 30, 33, 34, 37, 38, 39, 40 or 41.

6. The TdT variant according to claim 1, having said amino acid sequence at least ninety percent identical to an amino acid sequence selected from SEQ ID NO: 2, 3, 4, 5, 6, 7, 8, 9, 10, 11, 12, 13, 14, 15, 16, 18, 21, 23, 25, 27, 29, 31, 32, 35, or 36, wherein such selected amino acid sequence is subject to two or more mutations listed in Table 1 for its SEQ ID NO.

7. The TdT variant according to claim 1, said 3′-O-protected nucleotide comprises a 3′-O-protecting group selected from the group consisting of 3′-O-methyl, 3′-O-(2-nitrobenzyl), 3′-O-allyl, 3′-O-amine, 3′-O-azidomethyl, 3′-O-tert-butoxy ethoxy, 3′-O-(2-cyanoethyl), 3′-O-nitro, and 3′-O-propargyl.

8. A terminal deoxynucleotidyl transferase (TdT) variant comprising an amino acid sequence at least ninety percent identical to an amino acid sequence selected from SEQ ID NO: 42, 44, 46, 48, 50, 78 or 80 wherein the amino acid sequence of the TdT variant comprises a stabilizing amino acid at position 4 with respect to SEQ ID NO: 44, 78 and 80, a stabilizing amino acid at position 5 with respect to SEQ ID NO: 42, and a stabilizing amino acid at position 6 with respect to SEQ ID NO: 46, 48 and 50; and wherein the TdT variant (i) is capable of synthesizing a nucleic acid fragment without a template and (ii) is capable of incorporating a 3′-O-protected-nucleotide onto a free 3′-hydroxyl of a polynucleotide.

9. The TdT variant according to claim 8, wherein said amino acid sequence of said TdT variant is at least ninety percent identical to the amino acid sequence of SEQ ID NO: 42, SEQ ID NO: 78 or SEQ ID NO: 80 and comprises said stabilizing amino acid at position 4.

10. The TdT variant according to claim 8, wherein said stabilizing amino acid increases the melting temperature of said TdT variant by at least 1° C. relative to a melting temperature of a TdT of the same amino acid sequence except for said stabilizing amino acid substitution, wherein the melting temperature is determined by a fluorescence-based thermal shift assay comprising SYPRO Orange dye in a solution of 0.5 cacodylate KOH buffer pH 7.4.

11. The TdT variant according to claim 9, wherein said stabilizing amino acid is selected from the group consisting of E, S, D and N.

12. A method of synthesizing a polynucleotide having a predetermined sequence, the method comprising the steps of:
a) providing an initiator having a 3′-terminal nucleotide having a free 3′-hydroxyl;
b) repeating cycles of (i) contacting under elongation conditions the initiator or elongated fragments having free 3′-O-hydroxyls with a 3′-O-blocked nucleoside triphosphate and a TdT variant according to claim 1, so that the initiator or elongated fragments are elongated by incorporation of a 3′-O-blocked nucleoside triphosphate to form 3′-O-blocked elongated fragments, and (ii) deblocking the elongated fragments to form elongated fragments having free 3′-hydroxyls, until the polynucleotide is formed.

13. The method according to claim 12, wherein said 3′-O-blocked nucleoside triphosphate is a 3′-O—NH2-nucleoside triphosphate, a 3′-O-azidomethyl-nucleoside triphosphate, or a 3′-O-allyl-nucleoside triphosphate.

14. The method according to claim 12, wherein said initiator is attached to a solid support and comprises a cleavable nucleotide or a cleavable linkage at its 3′-end and wherein said polynucleotides having said predetermined sequences are released from the solid support by cleaving the cleavable nucleotide or the cleavable linkage.

15. A kit for performing a nucleotide incorporation reaction comprising:
a) a TdT variant according to claim 1, b) one or more 3′-O-protected nucleoside triphosphates, and c) optionally at least one initiator.

16. A terminal deoxynucleotidyl transferase (TdT) variant having a loop 2 region, wherein the TdT variant comprises a peptide affinity tag inserted into the loop 2 region, and wherein the TdT variant (i) is capable of synthesizing a nucleic acid fragment without a template and (ii) is capable of incorporating a 3′-O-protected-nucleotide onto a free 3′-hydroxyl of a polynucleotide.

17. The TdT variant according to claim 16, wherein said peptide affinity tag is H₂-H₁₀.

18. The TdT variant according to claim 17, wherein said peptide affinity tag is H₄-H₈.

19. The TdT variant according to claim 16, comprising an amino acid sequence at least ninety percent identical to an amino acid sequence selected from SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41, wherein a glutamine at position 4 of such amino acid sequence is substituted with a stabilizing amino acid.

20. The TdT variant according to claim 19, wherein said stabilizing amino acid is selected from the group consisting of E, S, D and N.

21. The TdT variant according to claim 16, comprising an amino acid sequence at least ninety percent identical to an amino acid sequence selected from SEQ ID NOs: 42, 44, 46, 48, or 50, wherein each of the amino acid sequences of SEQ ID NOs: 42, 44, 46, 48, and 50 have said peptide affinity tag inserted into said loop 2 region.

22. The TdT variant according to claim 16, comprising an amino acid sequence at least ninety percent identical to an amino acid sequence selected from SEQ ID NOs: 57 or 58.


SEQUENCE LISTING (81 sequences)

SEQ ID NO: 1 - protein, 510 aa, ARTIFICIAL SEQUENCE
  TdT full length mouse
MDPLQAVHLGPRKKRPRQLGTPVASTPYDIRFRDLVLFILEKKMGTTRRAFLMELARRKGFRVENELSDSVTHIVAENNSGSDVLEWLQLQNIKASSELELLDISWLIECMGAGKPVEMMGRHQLVVNRNSSPSPVPGSQNVPAPAVKKISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKRVFLEAESEEEIFAHLGLDYIEPWERNA

SEQ ID NO: 2 - protein, 362 aa, ARTIFICIAL SEQUENCE
  TdT truncated mouse
KISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKRVFLEAESEEEIFAHLGLDYIEPWERNA

SEQ ID NO: 3 - protein, 361 aa, ARTIFICIAL SEQUENCE
  TdT Bovine truncated
KISQYACQRKTTLNNYNHIFTDAFEILAENSEFKENEVSYVTFMRAASVLKSLPFTIISMKDTEGIPCLGDKVKCIIEEIIEDGESSEVKAVLNDERYQSFKLFTSVFGVGLKTSEKWFRMGFRSLSKIMSDKTLKFTKMQKAGFLYYEDLVSCVTRAEAEAVGVLVKEAVWAFLPDAFVTMTGGFRRGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSRQFERDIRRYATHERKMMLDNHALYDKTKRVFLKAESEEEIFAHLGLDYIEPWERNA

SEQ ID NO: 4 - protein, 361 aa, ARTIFICIAL SEQUENCE
  TdT Human truncated
KISQYACQRRTTLNNCNQIFTDAFDILAENCEFRENEDSCVTFMRAASVLKSLPFTIISMKDTEGIPCLGSKVKGIIEEIIEDGESSEVKAVLNDERYQSFKLFTSVFGVGLKTSEKWFRMGFRTLSKVRSDKSLKFTRMQKAGFLYYEDLVSCVTRAEAEAVSVLVKEAVWAFLPDAFVTMTGGFRRGKKMGHDVDFLITSPGSTEDEEQLLQKVMNLWEKKGLLLYYDLVESTFEKLRLPSRKVDALDHFQKCFLIFKLPRQRVDSDQSSWQEGKTWKAIRVDLVLCPYERRAFALLGWTGSRQFERDLRRYATHERKMILDNHALYDKTKRIFLKAESEEEIFAHLGLDYIEPWERNA

SEQ ID NO: 5 - protein, 358 aa, ARTIFICIAL SEQUENCE
  TdT Chicken 1 truncated
KVSQYSCQRKTTLNNCNKKFTDAFEIMAENYEFKENEIFCLEFLRAASVLKSLPFPVTRMKDIQGLPCMGDRVRDVIEEIIEEGESSRAKDVLNDERYKSFKEFTSVFGVGVKTSEKWFRMGLRTVEEVKADKTLKLSKMQRAGFLYYEDLVSCVSKAEADAVSSIVKNTVCTFLPDALVTITGGFRRGKKIGHDIDFLITSPGQREDDELLHKGLLLYCDIIESTFVKEQIPSRHVDAMDHFQKCFAILKLYQPRVDNSSYNMSKKCDMAEVKDWKAIRVDLVITPFEQYAYALLGWTGSRQFGRDLRRYATHERKMMLDNHALYDKRKRVFLKAGSEEEIFAHLGLDYVEPWERNA

SEQ ID NO: 6 - protein, 368 aa, ARTIFICIAL SEQUENCE
  TdT Possum truncated
TISQYACQRRTTINNHNQRFTDAFEILAKNYEFKENDDTCLTFMRAISVLKCLPFEVVSLKDTEGLPWIGDEVKGIMEEIIEDGESLEVQAVLNDERYQSFKLFTSVFGVGLKTADKWYRMGFRTLNKIRSDKTLKLTKMQKAGLCYYEDLIDCVSKAEADAVSLLVQDAVWTFLPDALVTITGGFRRGKEFGHDVDFLITSPGAEKEQEDQLLQKVTNLWKKQGLLLYCDLIESTFEDLKLPSRKIDALDHFQKCFLILKLYHHKEDKRKWEMPTGSNESEAKSWKAIRVDLVVCPYDRYAFALLGWSGSRQFERDLRRYATHEKKMMLDNHALYDKTKKIFLKAKSEEEIFAHLGLEYIQPSERNA

SEQ ID NO: 7 - protein, 362 aa, ARTIFICIAL SEQUENCE
  TdT truncated shrew
KISQYACQRRTTLNNHNHIFTDAFEILAENCEFRENEGSYVTYMRAASVLKSLPFSIISMKDTEGIPCLADKVKCVIEEIIEDGESSEVKAVLNDERYKSFKLFTSVFGVGLKTAEKWFRLGFRTLSGIMNDKTLKLTHMQKAGFLYYEDLVSCVTRAEAEAVGVLVKEAVWAFLPDAIVTMTGGFRRGKKVGHDVDFLITSPEATEEQEQQLLHKVITFWEKEGLLLYCDLYESTFEKLKMPSRKVDALDHFQKCFLILKLHRECVDDGTSSQLQGKTWKAIRVDLVVCPYECRAFALLGWTGSPQFERDLRRYATHERKMMLDNHALYDKTKRKFLSADSEEDIFAHLGLDYIEPWERNA

SEQ ID NO: 8 - protein, 362 aa, ARTIFICIAL SEQUENCE
  TdT truncated dog
KISQYACQRRTTLNNYNNVFTDAFEVLAENYEFRENEVFSLTFMRAASVLKSLPFTIISMKDTEGIPCLGDQVKCIIEEIIEDGESSEVKAVLNDERYQSFKLFTSVFGVGLKTSEKWFRMGFRTLSKIKSDKSLKFTPMQKAGFLYYEDLVSCVTRAEAEAVGVLVKEAVGAFLPDAFVTMTGGFRRGKKMGHDVDFLITSPGSTDEDEEQLLPKVINLWERKGLLLYCDLVESTFEKLKLPSRKVDALDHFQKCFLILKLHHQRVDGGKCSQQEGKTWKAIRVDLVMCPYERRAFALLGWTGSRQFERDLRRYASHERKMILDNHALYDKTKKIFLKAESEEEIFAHLGLDYIEPWERNA

SEQ ID NO: 9 - protein, 362 aa, ARTIFICIAL SEQUENCE; identical to SEQ ID NO: 7
  TdT TRUNC MOLE

SEQ ID NO: 10 - protein, 362 aa, ARTIFICIAL SEQUENCE
  TdT trunc Pika
KISQYACQRRTTLNNHNHIFTDAFEILAENYEFKENEGCYVTYMRAASVLKSLPFTIVSMKDTEGIPCLEDKVKSIMEEIIEEGESSEVKAVLSDERYQCFKLFTSVFGVGLKTSEKWFRMGFRSLSNIRLDKSLKFTQMQKAGFRYYEDIVSCVTRAEAEAVDVLVNEAVRAFLPDAFITMTGGFRRGKKIGHDVDFLITSPELTEEDEQQLLHKVMNLWEKKGLLLYHDLVESTFEKLKQPSRKVDALDHFQKCFLIFKLYHERVGGDRCRQPEGKDWKAIRVDLVMCPYECHAFALLGWTGSRQFERDLRRYASHERKMILDNHALYDKTKRVFLQAENEEEIFAHLGLDYIEPWERNA

SEQ ID NO: 11 - protein, 365 aa, ARTIFICIAL SEQUENCE
  TRUNC HEDGEHOG
KISQYACQRRTSLNNCNHIFTDALDILAENHEFRENEVSCVAFMRAASVLKSLPFTIISMKDTKGIPCLGDKAKCVIEEIIEDGESSEVKAILNDERYQSFKLFTSVFGVGLKTSEKWFRMGFRTLNKIMSDKTLKLTRMQKAGFLYYEDLVSCVAKAEADAVSVLVQEAVWAFLPDAMVTMTGGFRRGKKLGHDVDFLITSPGATEEEEQQLLPKVINFWERKGLLLYHDLVESTFEKLKLPSRKVDALDHFQKCFLILKLHLQHVNGVGNSKTGQQEGKNWKAIRVDLVMCPYERRAFALLGWTGSRQFERDLRRFATHERKMMLDNHALYDKTKRIFLKAESEEEIFAHLGLDYIDPWERNA

SEQ ID NO: 12 - protein, 362 aa, ARTIFICIAL SEQUENCE
  truncated tree shrew
KISQYACQRRTTLNNCNRVFTDAFETLAENYEFRENEDSSVIFLRAASVLRSLPFTITSMRDTEGLPCLGDKVKCVIEEIIEDGESSEVNAVLNDERYKSFKLFTSVFGVGLKTSEKWFRMGFRTLSRVRSDKSLHLTRMQQAGFLYYEDLASCVTRAEAEAVGVLVKEAVGAFLPDALVTITGGFRRGKKTGHDVDFLITSPGSTEEKEEELLQKVLNLWEKKGLLLYYDLVESTFEKLKTPSRKVDALDHFPKCFLILKLHHQRVDGDKPSQQEGKSWKAIRVDLVMCPYERHAFALLGWTGSRQFERDLRRYATHERKMMLDNHALYDKTKRVFLKAESEEDIFAHLGLDYIEPWERNA

SEQ ID NO: 13 - protein, 375 aa, ARTIFICIAL SEQUENCE
  TRUNCATED PLATYPUS
QVSQYACERRTTLNNCNQKFTDAFEILAKDFEFRENEGICLAFMRAISVLKCLPFTIVRMKDIEGVPWLGDQVKSIIEEIIEDGESSSVKAVLNDERYRSFQLFNSVFEVGLTDNGENGIARGFQTLNEVITDENISLTKTTLSTSLWNYLPGFLYYEDLVSCVAKEEADAVYLIVKEAVRAFLPEALVTLTGGFRRGKKIGHDVDFLISDPESGQDEQLLPNIIKLWEKQELLLYYDLVESTFEKTKIPSRKVDAMDHFQKCFLILKLHHQKVDSGRYKPPPESKNHEAKNWKAIRVDLVMCPFEQYAYALLGWTGSRQFERDLRRYATHEKKMMLDNHALYDKTKKIFLKAESEEDIFTHLGLDYIEPWERNA

SEQ ID NO: 14 - protein, 365 aa, ARTIFICIAL SEQUENCE
  B10 trunc wt (Serinus canaria)(canary)
KVSQYSCQRKTTLNNYNKKFTDAFEVMAENYEFKENEIFCLEFLRAASLLKSLPFSVTRMKDIQGLPCMGDQVRDVIEIIEEGESSRVKEVLNDERYKAFKQFTSVFGVGVKTSEKWYRMGLRTVGEVKADKTLKLSKMQKAGFLYYEDLVSCVSKAEADAVSLIVKNTVCTFLPDALVTITGGFRRGKNIGHDIDFLITNPGPREDDELLHKVIDLWKKQGLLLYCDIIESTFVKEQLPSRKIDAMDHFQKCFAILKLYQPRVDNSTCNTSKKLEMAEVKDWKAIRVDLVITPFEQYSYALLGWTGSRQFGRDLRRYAAHERRMILDNHGLYDRTKRIFLKAGSEEEIFAHLGLDYVEPWERNA

SEQ ID NO: 15 - protein, 366 aa, ARTIFICIAL SEQUENCE
  B19 trunc wt (Neopelma chrysocephalum) (saffron-crested neopelma)
KVSQYSCQRKTTLNNYNKKFTDAFEIMAENYEFKENEIFCLEFLRAASLLKYLPFPVTRMKDIQGLPCIGDQVRDVIEGIIEEGESSRVKEVLNDERYKAFKQFTSVFGVGVKTSEKWYRMGLRTVEELKADKTLKLSKMQKAGFLYYEDLVSCVSKAEADAVTLIVKNTVSTFLPDALVTITGGFRRGKKMGHDIDFLITNPGPREDDELLHKVVDLWKKQGLLLYCDIIESTFVEEQLPSRKVDAMDNFQKCFTILKLYQPGVDNSSYNMSKKSDMAEVKDWKAIRVDLVITPFEQYAYALLGWTGSREFGRDLRRYASHERKMILDNHGLYDRRKRIFLKAGSEEEIFAHLGLDYVEPWERNA

SEQ ID NO: 16 - protein, 366 aa, ARTIFICIAL SEQUENCE
  B57 trunc wt (Alligator sinensis)
KVSQYACQRRTTLNNYNKKFTDAFEILAENCEFRENRLGCLEFLRAASVLKFLPFPIVKMKNIEGLPCMGDKVKCVIEEILEEGESCQAKEILNDERYKSFKLFTSVFGVGLKTTEKWYRMGFRTLEEVKAEKTLKLSRMQIAGFLHYEDIISYVSKAEADAVSLLIKDTVCMFLPDALVTITGGFRRGKKTGHDVDFLITNPGPEEEKELLHKVVDLWEKQGLLLYYDVIESTFEKEKRPSRKVDALDHFQKCFAILKLHQQRRGNSNSNISKESDKAEVKDWKAIRVDLVISPFEQYAYALLGWTGSRQFERDLRRYASRERKMMLDNHALYDKTKRTFLKAESEEEIFAHLGLDYIEPWERNA

SEQ ID NO: 17 - protein, 378 aa, ARTIFICIAL SEQUENCE
  B57 trunc new
KISQYACQRRTTLNNYNKKFTDAFEILAENYEFNENKGFCLAFRRAASVLKFLPFTIVRMNDIQGLPWMGDQVKRVIEEILEEGESSKVKEVLNDEKYKAFKLFTSVFGVGRKTSEKWYRMGLRTLEEVKADKTLKLTKMQKAGFLYYEDLISRVSKAEADAVSLIVKDTVWKFLPDALVTLTGGFRLGKKMGHDVDFLITTPGPEEEEELLHKVIDLWKKQGLLLYYDIIESTFEKTKLPSRTVDALDHFQKCFAILKLHKQRVDNGNSNQSKEFDKEETKDWKAIRVDLVITPFEQYAYALLGWTGSAFFNRDLRRYATHEKKMMLDNHALYDKTKRIFLKAASEEEIFAHLGLDYLEPWERNAAAAIRLLTPDRS

SEQ ID NO: 18 - protein, 363 aa, ARTIFICIAL SEQUENCE
  B59 trunc wt (Xenopus laevis)
KVSQYACQRCTTLQDTNRIFTDAFDILAEHFEFCENKGRTVAFLRASSLIKSLPFPITAMKELEGLPWLGDQMKGIIEEILEEGKSYKVLEVMNEERYKSFKQFTSVFGVGLKTSDKWFRMGFRTLEEIKNEKELKLTKMQKCGLLYYEDITSYVSRAEAETTEQLIKSIVWKFVPDAIVTLTGGFRRGKKKGHDVDILITCARKGKEKNILHNTMSVLKNRGLLLFYNIIESTFDETKLPSRHVDALDHFQKCFTILKLPKRQMDIGNIIDPHECERKNWKAVRLDLVITPYEQYPYALLGWTGSRQFERDLRRYATHEKRMMLDNHGLYDKTKNNFLKANNEEDIFKQLGLDYLEPWERNA

SEQ ID NO: 19 - protein, 376 aa, ARTIFICIAL SEQUENCE
  Xenopus  B59 trunc new
KVSQYACQRRTTLQDTNRIFTDAFDILAENYEFNENKGPCVAFRRASSVLKSLPFPIVAMKDLEGLPWLGDQMKRIIEEILEEGKSSKVVEVMNDERYKAFKLFTSVFGVGRKTSEKWFRMGFRTLEEIKSDKELKLTKMQKAGLLYYEDITSAVSKAEAEAVEQLIKSIVWKFVPDAIVTLTGGFRLGKKMGHDVDILITTPRKGEKEEILHKTINVLKKRGLLLFYNIIESTFDETKLPSRHVDALDHFQKCFTILKLQKQQLDNGNSNEPFETEKKNWKAIRVDLVITPYEQYAYALLGWTGSPQFNRDLRRYATHEKRMMLDNHALYDKTKNIFLKANSEEDIFTHLGLDYLEPWERNAAAAIRLLTKPERS

SEQ ID NO: 20 - protein, 380 aa, ARTIFICIAL SEQUENCE
  Notechis  B63 trunc new
SISQYACQRRTTLNNYNKKFTDAFEILAENYEFNENKGFCLAFRRAASVLKFLPFTIVRVNDIEGLPWMGEQVKRIIEDILEEGESSKVKAVLNNENYRAFKLFTSVFGVGRKTSEKWYRMGLRTLEEVKADKNLKLTRMQKAGFLHYEDLISRVSKAEADAASLIVKDTVWKFLPNALVTLTGGFRLGKKMGHDVDFLITVPGSRQEEEELLHTVIDLWKKQGLLLYYDLIESTFEKTKLPSRTVDALDHFQKCFAILKLHKERVDKGNSIQSKAFAEEETKDWKAIRVDLVVTPFEQYAFALLGWTGSPFFNRDLRRYATHEKKMMLDNHALYDKTKKIFLKAASEEEIFAHLGLDYLEPWERNAAAAIRLLTKPERS

SEQ ID NO: 21 - protein, 363 aa, ARTIFICIAL SEQUENCE
  F15 trunc wt (Salmo trutta) brown trout
NVSQYACLRRTTTENHNKIFTDVLEELAENSEFNESKGPCLAFRRAASVLKSLPSAVHCLGAIQGLPCLGEHTKAVMEEILIFGRSFKVEEVQSDERYQALKLFTSVFGVGPKTAEKWYRRGLRSLKEILAEPNIQLNRMQRAGFLYYRDISKAVSKAEAKALSSIIEETAHWIAPDSILALTGGFRRGKEYGHDVDFLLTMPEMGKEEGLLLRVIDRLRDQGILLYCEHQDSTFDMSKLPSHRFEAMDHFEKCFLILRLEEGQVEGDGGLQKDPGESRGWRAVRVDLVAPPVDRYAFVLLGWTGSRQFERDLRRFASKERGMCLDNHALYDKTKKLFLPATSEEDIFAHLGLEYVEPWQRNA

SEQ ID NO: 22 - protein, 377 aa, ARTIFICIAL SEQUENCE
  F15 trunc new
  misc_feature (372)..(372): Xaa can be any naturally occurring amino acid
TVSQYACQRRTTMENHNKIFTDAFEVLAENSEFNESKESRDAFRRAASVLKSLPSAVRSLGDTEGLPCLGEHTKRVIEEILEYGRSSKVEDILSDERYQTMKLFTSVFGVGPKTAEKWYRRGLRSLEEVRAEPSIHLNRMQRAGFLYYRDISKRVSKAEAKALGNIIEETVHAIAPDAIVTLTGGFRLGKEFGHDVDFILTTPEMGKEEGLLPRVIDRLRNQGILLYCDYQESTFDMSKLPSRTFEAMDHFQKCFLIIKLKEGLVEGEGGLQSDPGDRRGWRAVRVDLVAPPVDRYAFALLGWTGSPQFNRDLRRFARLERRMLLDNHALYDKTKKIFLPAKTEEDIFAHLGLEYIEPWQRNAAAAIRLLTXPRLRK

SEQ ID NO: 23 - protein, 360 aa, ARTIFICIAL SEQUENCE
  F24 trunc wt (Electrophorus electricus) electric eel
TVSQYACQRRTTLDNHNKVFTDALEVLIENYEFSDNKGACVGFRRAASVLKSLPKPLRCLKDMEGLPCLGDDTKAIIEEIYECGSSSRVENILSDEKYQTLKLFTSVFGVGPKTGEKWYRRGLRALEQVHSEPSIQLNKMQAAGFLYYEDISKPVSRAEAKAVGCIIEEVASCFSSSVTITLTGGFRRGKEFGHDVDFLLSIPEPGKEDGLLPAVIDRLRKQGILLYSDLQESTLQQWKRPSRCFDSMDHFQKCFLIVKLWTRLVEGHREDPSSQRDWKAVRVDLVVPPVDCYAFALLGWSGSTQFERDLRRFARLERRMLLDNHALYDKTTNTFLQAKTEEDIFAHLGLDYIEPWQRNA

SEQ ID NO: 24 - protein, 373 aa, ARTIFICIAL SEQUENCE
  F24 trunc new
TVSQYACQRRTTLDNHNKIFTDALEVLAENYEFSDSKESRDAFRRAASVLKSLPTPLRSLQDTEGLPCLGEETKRVIEEIFEEGSSSRVEDILSDERYQTLKLFTSVFGVGPKTAEKWYRRGLRSLEQVRSDPSIHLNRMQTAGFLYYEDISKPVSKAEAKALGNIIEETASAFSPSVTITLTGGFRLGKEFGHDVDFLLTVPEPGKEDGLLPAVIDRLRSQGILLYSDFQESTFDLSKLPSRRFEAMDHFQKCFLIVKLKAGLVEGEGADPGDRRDWRAVRVDLVAPPVDRYAFALLGWSGSAFFNRDLRRFARLERGMLLDNHALYDKTTNTFLQAKTEEDIFAHLGLDYIEPWQRNAAAAIRLLTKPERS

SEQ ID NO: 25 - protein, 361 aa, ARTIFICIAL SEQUENCE
  F32 trunc wt (Anabas testudineus) walking fish
TVSQYACQRRTTTENNNKILTDAFEVLAESYELNQLEGPCLAFRRAASVLKSLPWAVQCLGATQGLPCLGEHTKALIEEILQYGHSFEVEKILSDERYQTLKLFTSVFGVGPKTAEKWYRRGLRSFSDILAEPSIQLNRMQQSGFLHYGDISRAVSKAEARALGNIIDEAVHAITPDGILALTGGFRRGKEFGHDVDFIVTTPEQGKEETLLPNIIDRLKEQGILLYSDYQTSTFDISKLPSHKFEAMDHFAKCFLILRLEGSLVDRGLNSTEGDSRGWRAVRVDLVSPPMERYAYALLGWTGSRQFERDLRRFARLEQHMLLDNHALYDKTKKEFLAATTERDIFAHLGLEYIEPWQRNA

SEQ ID NO: 26 - protein, 375 aa, ARTIFICIAL SEQUENCE
  F32 trunc new  walking fish
TVSQYACQRRTTTENNNKIFTDAFEVLAESYEFNEMEESRDAFRRAASVLKSLPWAVQSLGATQDLPCLGEHTKRVMEEILQYGRSFEVEKILSDERYQTLKLFTSVFGVGPKTAEKWYRRGLRSFEDIRAEPSIHLNRMQQSGFLHYGDISRAVSKAEARALGNIIDEAVHAITPDAILALTGGFRLGKEFGHDVDFIVTTPELGKEESLLPNIIDRLKKQGILLYSDYQASTFDMSKLPSHRFEAMDHFAKCFLILRLEGSQVEGGLNSAEGDSRGWRAVRVDLVSPPMDRYAFALLGWTGSAFFNRDLRRFARMERRMLLDNHALYDKTKKEFLAATTEKDIFAHLGLEYIEPWQRNAAAAIRLLTLPRLRQ

SEQ ID NO: 27 - protein, 361 aa, ARTIFICIAL SEQUENCE
  F57 trunc wt (Poecilia reticulate) guppy
KVSQYACQRRTTVENNNRIFTDAFEVLAENYEFNEIEGRCLAFRRAASVLKSLPWAVRSVGATLDLPCLGEHTTAVMKEILQYGRSFEVEKILSDERCQTLKLFTSVFGVGPKTAEKWYRRGLRSFSDVLAQPGIHLNRMQQSGFLHYGDISRAVSKAEARAVGNIIDEAVHVITPNAILALTGGFRRGKDFGHDVDFIVTTTELGKEKNLLISVIESLKKQGLLLFSDYQASTFDISKLPSHRFEAMDHFAKCFLILRLEGSRVEGGLQRAQADGRGWRAVRVDLVSPPADRFAFTMLGWTGSRMFERDLRRFARLERQMLLDNHALYDKTKKEFLTAATEKDIFDHLGLEYIEPWQRNA

SEQ ID NO: 28 - protein, 378 aa, ARTIFICIAL SEQUENCE; identical to SEQ ID NO: 17
  F57 trunc new (Poecilia reticulate) guppy

SEQ ID NO: 29 - protein, 362 aa, ARTIFICIAL SEQUENCE
  N100 trunc wt (short Rattus norvegicus)
KISQYACQRRTTLNNHNQLFTDAFDILAENYEFRENEVSCLPFMRAASVLKSLSFPIVSMKDIEGIPCLGDKVKCVIEGIIEDGESSEVKAVLNDERYKSFKLFTSVFGVGLKTAEKWFRMGFRTLSKIKSDKSLRFTHMQKAGFLYYEDLVSCVNRAEAEAVSMLVKEAVVAFLPDALVTMTGGFRRGKMTGHDVDFLITSPEATEEEEQQLLHKVTNFWRQQGLLLYCDIIESTFEKFKLPSRKVDALDHFQKCFLILKLHRGLVRSEESGQQEGKDWKAIRVDLVMCPYERRAFALLGWTGSRQFERDLRRYATHERKMMLDNHALYDKTKRVFLEAESEEEIFAHLGLDYIEPWERNA

SEQ ID NO: 30 - protein, 380 aa, ARTIFICIAL SEQUENCE
  N100 trunc new
SVVQKISEYACQRRTTLNNHNEVFTRAFDILAENYEFRENEESRDAFRRASSVLKSLPFPIISMKDTEGIPCLGDKVKRVIEEIIEDGESSEVKAVLNDERYKAFKLFTSVFGVGRKTAEKWFRMGFRTLEKIKSDKSLRFTQMQKAGFLYYEDLAERVNRAEAEAIEMLVKEAVVAFLPDALVTMTGGFRLGKMTGHDVDFLITSPEATEEEEQQLLHKVTDFWRQQGLLLYCDIIESTFEKFKLPSRTVDALDHFQKCFLILKLHHGRVVSEKSGQQEGKGWKAIRVDLVMCPYERRAFALLGWTGSPFFNRDLRRYATHERKMMLDNHALYDKTKRVFLEAESEEEIFAHLGLDYIEPWERNAAAAIRLLTKPERSD

SEQ ID NO: 31 - protein, 362 aa, ARTIFICIAL SEQUENCE
  N14 trunc wt (Piliocolobus tephrosceles)
KISQYACQRRTTLNNCNQIFTDAFDILAENCEFRENEDSCVTFMRAASVLKSLPFTIISMKDTEGIPCLGSKVKCIIEEIIEDGESSEVKAVLNDERYQSFKLFTSVFGVGLKTSEKWFRMGFRTLSKVRSDESLKFTRMQRAGFLYYEDLVSCVTRAEAEAVSALVKEAVWAFLPDAFVTMTGGFRRGKKMGHDVDFLITSPGSTEDEEQQLLQKVMNLWEKKGLLLYYDLVESTFEKLRLPSRKVDALDHFQKCFLIFKLPLQRVDSDQSSWQGGKTWKAIRVDLVMCPYERRAFALLGWTGSRQFERDLRRYATHERKMILDNHALYDKTKRIFLKAESEEEIFAHLGLDYIEPWERNA

SEQ ID NO: 32 - protein, 362 aa, ARTIFICIAL SEQUENCE
  N27 trunc wt (Sus scrofa) pig
KISQYACQRRTTLNNCNHIFTDAFEVLAENYEFRENETFCLAFMRAASVLKSLPFTIISMKDTEGIPCLGDKVKCVIEEIIEDGESSEVKAVLNDERYQSFKLFTSVFGVGLKTSERWFRMGFRSLSKIRSDKTLKFTRMQKAGFLYYEDLVSCVTRAEAEAVGVLVKEAVQAFLPDAFVTMTGGFRRGKKMGHDVDFLITSPGSTDDEEQQLLPKVVNLWEREGLLLYCDLVESTLEKSKLPSRNVDALDHFQKCFLILKLHHQRVDSGMSSQQEGKTWKAIRVDLVMCPYELRAFALLGWTGSRQFERDLRRYATHERKMILDNHALYDKTKRIFLKAESEEEIFAHLGLDYLEPWERNA

SEQ ID NO: 33 - protein, 375 aa, ARTIFICIAL SEQUENCE
  N27 trunc new  pig
KISQYACQRRTTLNNYNEIFTRAFEILAENYEFRENEESYVTFRRAASVLKSLPFTIISMKDTEGIPCLGDKVKRVIEEIIEDGESSEVKAVLNDERYKAFKLFTSVFGVGRKTSEKWFRMGFRTLEKIRSDKTLKFTRMQKAGFLYYEDLVSRVTRAEAEAVSVLVKEAVWAFLPDAFVTMTGGFRLGKKIGHDVDFLITSPGSTEEEEQQLLHKVINLWEKKGLLLYYDLVESTFEKLKLPSRTVDALDHFQKCFLILKLHHQRVGSGKSSQQEGKTWKAIRVDLVMCPYERRAFALLGWTGSPQFNRDLRRYATHERKMMLDNHALYDKTKRIFLKAESEEEIFAHLGLDYIEPWERNAAAAIRLLTKPERS

SEQ ID NO: 34 - protein, 368 aa, ARTIFICIAL SEQUENCE
  N35 trunc new
KISQYACQRRTTLNNYNEIFTRAFEVLAENYEFRENEESYVTFRRAASVLKSLPFTIISMKDTEGIPCLGDKVKRVIEEIIEDGESSEVKAVLNDERYQAFKLFTSVFGVGRKTSEKWFRMGFRTLEKIRSDKTLKFTRMQKAGFLYYEDLAERVTRAEAEAVSVLVKEAVWAFLPDAFVTMTGGFRLGKKIGHDVDFLITSPGSTEEEEQQLLPKVINLWERKGLLLYYDLVESTFEKLKLPSRKVDALDHFQKCFLILKLHHQRVGSGKSSQQEGKTWKAIRVDLVMCPYERRAFALLGWTGSAFFNRDLRRYATHERKMILDNHALYDKTKRIFLKAESEEEIFAHLGLDYIEPWERNAAAAIGC

SEQ ID NO: 35 - protein, 361 aa, ARTIFICIAL SEQUENCE
  N72 trunc wt (Bubalus bubalis) water buffalo
KISQYACQRKTTLNNYNHIFTDAFEILAENSEFKENEVSYVTFMRAASVLKSLPFTIISMKDTQGIPCLGDKVKCVIEEIIEDGESSEVKAVLNDERYQSFKLFTSVFGVGLKTSEKWFRMGFRSLSKITSDKTLKFTKMQKAGFLYYEDLVSCVTRAEAEAVGVLVKEAVWAFLPDAFITMTGGFRRGKKIGHDVDFLITSPGSAEDEEQLLPKVINLWEKKGLLLYYDLVESTFEKFKLPSRQVDTLDHFQKCFLILKLHHQRVDSGRSNQQEGKTWKAIRVDLVMCPYENRAFALLGWTGSRQFERDIRRYATHERKMMLDNHALYDKTKRVFLKAESEEEIFAHLGLDYIEPWERNA

SEQ ID NO: 36 - protein, 362 aa, ARTIFICIAL SEQUENCE
  N93or trunc wt (Marmota flaviventris) marmot
TISQYACQRRTTLNNCNRVFTDAFDVLAENYEFRENESCSVVFMRAASVLKSLPFTIISMRDLEGIPCLEGKAKSIIEEIIEDGESSEVKAVLNDERYKSFKLFTSVFGVGLKTSEKWFRMGFRTLSKIRSDKSLKFTHMQKAGFLYYEDLVSCVTRAEAEAVSVLVKEAVWAFLPDAFITMTGGFRRGKNIGHDVDFLITSAEATEEEEQQLLHKVTNLWEKKGLLLYCDLVESTFEKLKTPSRKVDALDHFQKCFLILKLHHQRVDSDKASQQGGKNWKAIRVDLVMCPYERRAFALLGWTGSRQFERDLRRYATHERKMILDNHALYDKTKRIFLKAESEEEIFAHLGLDYIEPWERNA

SEQ ID NO: 37 - protein, 362 aa, ARTIFICIAL SEQUENCE
  Truncated mouse TdT
KISQYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLAERVNRPEAEAIRMLVKEAVVTFLPDALVTMTGGFRLGKMTGHDVDFLITSPEATEDEEQQLLHKVTDFWKQQGLLLYCDILESTFEKFKQPSRTVDALDHFQKCFLILKLDHPRVHSVKSGQQEGKGWKAIRVDLVMCPYDRRAFALLGWTGSPQFNRDLRRYATHERKMMLDNHALYDKTKRVFLEAESEEEIFAHLGLDYIEPWERNA

SEQ ID NO: 38 - protein, 361 aa, ARTIFICIAL SEQUENCE
  bovine-mouse chimera
KISQYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSPQFNRDLRRYATHERKMMLDNHALYDKTKRVFLEAESEEEIFAHLGLDYIEPWERNA

SEQ ID NO: 39 - protein, 362 aa, ARTIFICIAL SEQUENCE
  bovine-mouse chimera
  misc_feature (210)..(211): Xaa can be any naturally occurring amino acid
KISQYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLAERVNRPEAEAIRMLVKEAVVTFLPDALVTMTGGFRLGKMTGHDVDFLITSPGSAEDEXXQLLPKVINLWEKKGLLLYYDLVESTFEKFKLPSRQVDTLDHFQKCFLILKLHHQRVDSSKSNQQEGKTWKAIRVDLVMCPYENRAFALLGWTGSPQFNRDLRRYATHERKMMLDNHALYDKTKRVFLEAESEEEIFAHLGLDYIEPWERNA

SEQ ID NO: 40 - protein, 362 aa, ARTIFICIAL SEQUENCE
  mouse M27
KISQYACQRRTTLNNYNQLFTDALDILAENDEFRENEESCLAFRRASSVLKSLPFPITSMKDTEGIPCLGDKVKSIIEGIIEDGESSEVKAVLNDERYKSFKLFTSVFGVGLKTAEKWFRMGFRTLSKIQSDKSLRFTQMQKAGFLYYEDLVSGVNRPEAEAVSMLVKEAVVTFLPDALVTMTGGFRLGKMTGHDVDFLITSPEATEDEEQQLLHKVTDFWKQQGLLLYCDILESTFEKFKQPSRTVDALDHFQKCFLILKLDHPRVHSVKSGQQEGKGWKAIRVDLVMCPYDRRAFALLGWTGSPQFNRDLRRYATHERKMMLDNHALYDKTKRVFLEAESEEEIFAHLGLDYIEPWERNA

SEQ ID NO: 41 - protein, 361 aa, ARTIFICIAL SEQUENCE
  M54
KISQYACQRKTTLNNYNEIFTRAFEILAENSEFKENEESYVTFRRAASVLKSLPFTIISMKDTEGIPCLGDKVKRIIEEIIEDGESSEVKAVLNDERYQAFKLFTSVFGVGRKTSEKWFRMGFRSLEKIRSDKTLKFTKMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSAFFNRDIRRYATHERKMMLDNHALYDKTKRVFLKAESEEEIFAHLGLDYIEPRERNA

SEQ ID NO: 42 - protein, 362 aa, ARTIFICIAL SEQUENCE
  TdT
KKISEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSPQFNRDLRRYATHERKMMLDNHALYDKTKRVFLEAESEEEIFAHLGLDYIEPWERNA

SEQ ID NO: 43 - protein, 377 aa, ARTIFICIAL SEQUENCE
  TdT
MASSHHHHHHSSGSEKKISEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSPQFNRDLRRYATHERKMMLDNHALYDKTKRVFLEAESEEEIFAHLGLDYIEPWERNA

SEQ ID NO: 44 - protein, 361 aa, ARTIFICIAL SEQUENCE
  TdT
KISEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSPQFNRDLRRYATHERKMMLDNHALYDKTKRVFLEAESEEEIFAHLGLDYIEPWERNA

SEQ ID NO: 45 - protein, 376 aa, ARTIFICIAL SEQUENCE
  TdT
MASSHHHHHHSSGSEKISEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSPQFNRDLRRYATHERKMMLDNHALYDKTKRVFLEAESEEEIFAHLGLDYIEPWERNA

SEQ ID NO: 46 - protein, 363 aa, ARTIFICIAL SEQUENCE
  TdT
VTTVSEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSPQFNRDLRRYATHERKMMLDNHALYDKTKRVFLEAESEEEIFAHLGLDYIEPWERNA

SEQ ID NO: 47 - protein, 379 aa, ARTIFICIAL SEQUENCE
  TdT
MASSHHHHHHSSGSENVTTVSEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSPQFNRDLRRYATHERKMMLDNHALYDKTKRVFLEAESEEEIFAHLGLDYIEPWERNA

SEQ ID NO: 48 - protein, 363 aa, ARTIFICIAL SEQUENCE
  ii-ell-40 without His tag
VATVSEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSPQFNRDLRRYATHERKMMLDNHALYDKTKRVFLEAESEEEIFAHLGLDYIEPWERNA

SEQ ID NO: 49 - protein, 379 aa, ARTIFICIAL SEQUENCE
  ii-ell-40 with His tag
MASSHHHHHHSSGSENVATVSEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSPQFNRDLRRYATHERKMMLDNHALYDKTKRVFLEAESEEEIFAHLGLDYIEPWERNA

SEQ ID NO: 50 - protein, 363 aa, ARTIFICIAL SEQUENCE; identical to SEQ ID NO: 48
  TdT

SEQ ID NO: 51 - protein, 379 aa, ARTIFICIAL SEQUENCE; identical to SEQ ID NO: 49
  TdT

SEQ ID NO: 52 - DNA, 18 bp, ARTIFICIAL SEQUENCE
  primer
aaaaaaaaaaaaaagggg

SEQ ID NO: 53 - DNA, 12 bp, ARTIFICIAL SEQUENCE
  primer
cagttaaaaact

SEQ ID NO: 54 - DNA, 11 bp, ARTIFICIAL SEQUENCE
  primer
gagttaaaact

SEQ ID NO: 55 - DNA, 10 bp, ARTIFICIAL SEQUENCE
  primer
cagcaaggct

SEQ ID NO: 56 - protein, 25 aa, ARTIFICIAL SEQUENCE
  peptide affinity tag
MASSHHHHHHSSGSENLYFQTGSSG

SEQ ID NO: 57 - protein, 365 aa, Artificial Sequence
  45-751
MAEKKISEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEEIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTLEKFKLPSRQVDTLDHFQKCFLILKLHRQRVDSSKHHHHHHKTWKAIRVDLVMCPYENRAFALLGWTGSPQFNRDLRRYATHERKMMLDNHALYDKTKRVFLEAESEEEIFAHLGLDYIEPWERNA

SEQ ID NO: 58 - protein, 369 aa, Artificial Sequence
  46-737
MAHHGSEKKISEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEEIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTLEKFKRPSRQVDTLDHFQKCFLILKLHRQRVDSSKHHHHHHETWKAIRVDLVMCPYENRAFALLGWTGSPQFNRDLRRYATHERKMMLDNHALYDKTKRVFLEAESEEEIFAHLGLDYIEPWERNA

SEQ ID NO: 59 - protein, 6 aa, Artificial Sequence
  Poly His
HHHHHH

SEQ ID NO: 60 - protein, 8 aa, Artificial Sequence
  FLAG
DYKDDDDK

SEQ ID NO: 61 - protein, 4 aa, Artificial Sequence
  DYKD
DYKD

SEQ ID NO: 62 - protein, 10 aa, Artificial Sequence
  c-myc
EQKLISEEDL

SEQ ID NO: 63 - protein, 8 aa, Artificial Sequence
  Strep II
WSHPQFEK

SEQ ID NO: 64 - protein, 5 aa, Artificial Sequence
  Poly Arg
RRRRR

SEQ ID NO: 65 - protein, 6 aa, Artificial Sequence
  AU1 epitope
DTYRYI

SEQ ID NO: 66 - protein, 6 aa, Artificial Sequence
  AU5 epitope
TDFYLK

SEQ ID NO: 67 - protein, 11 aa, Artificial Sequence
  Bacteriophage T7 epitope
MASMTGGQQMG

SEQ ID NO: 68 - protein, 14 aa, Artificial Sequence
  Bacteriophage V5 epitope
GKPIPNPLLGLDST

SEQ ID NO: 69 - protein, 6 aa, Artificial Sequence
  Bluetongue virus tag
QYPALT

SEQ ID NO: 70 - protein, 10 aa, Artificial Sequence
  E2 epitope
SSTSSDFRDR

SEQ ID NO: 71 - protein, 6 aa, Artificial Sequence
  EE-tag
EYMPME

SEQ ID NO: 72 - protein, 11 aa, Artificial Sequence
  HSV epitope
KPPTPPPEPET

SEQ ID NO: 73 - protein, 5 aa, Artificial Sequence
  Poly Asp
DDDDD

SEQ ID NO: 74 - protein, 11 aa, Artificial Sequence
  Poly Phe
FFFFFFFFFFF

SEQ ID NO: 75 - protein, 4 aa, Artificial Sequence
  Poly Cys
CCCC

SEQ ID NO: 76 - protein, 9 aa, Artificial Sequence
  S1 tag
NANNPDWDF

SEQ ID NO: 77 - protein, 15 aa, Artificial Sequence
  S tag
KETAAAKFERQHMDS

SEQ ID NO: 78 - protein, 361 aa, Artificial Sequence
  TdT-M96 without N-terminal His tag
KISEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEEIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYNLVESTFEKFKLPSRQVDTLDHFQKCFLILKLHHQRVDSSKSNQQEGKTWKAIRVDLVMCPYENRAFALLGWTGSPQFNRDLRRYATHERKMMLDNHALYDKTKRVFLEAESEEEIFAHLGLDYIEPWERNA

SEQ ID NO: 79 - protein, 377 aa, Artificial Sequence
  TdT-M96 with N-terminal His tag
MASSHHHHHHSSGSEKKISEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEEIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYNLVESTFEKFKLPSRQVDTLDHFQKCFLILKLHHQRVDSSKSNQQEGKTWKAIRVDLVMCPYENRAFALLGWTGSPQFNRDLRRYATHERKMMLDNHALYDKTKRVFLEAESEEEIFAHLGLDYIEPWERNA

SEQ ID NO: 80 - protein, 361 aa, Artificial Sequence
  TdT-M103 without N-terminal His tag
KISEYACQRRTTLNNYNELFTRALDILVENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLRESTFEKFKLPSRQVDTLDHFQKCFLILKLHHQRVDSSKSNQQEGKTWKAIRVDLVMCPYENRAFALLGWTGSPQFNRDLRRYATHERKMMLDNHALYDKTKRVFLEAESEEEIFAHLGLDYIEPWERNA

SEQ ID NO: 81 - protein, 377 aa, Artificial Sequence
  TdT-M103 with N-terminal His tag
MASSHHHHHHSSGSEKKISEYACQRRTTLNNYNELFTRALDILVENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLRESTFEKFKLPSRQVDTLDHFQKCFLILKLHHQRVDSSKSNQQEGKTWKAIRVDLVMCPYENRAFALLGWTGSPQFNRDLRRYATHERKMMLDNHALYDKTKRVFLEAESEEEIFAHLGLDYIEPWERNA